(19) United States
(12) Patent Application Publication
(10) Pub. No.: US 2023/0183313 A1
(43) Pub. Date: Jun. 15, 2023

(54) ISOLATED CHIMERIC ANTIGEN RECEPTOR, MODIFIED T CELL COMPRISING SAME AND USE THEREOF

(71) Applicants: Jiangsu Hengrui Medicine Co., Ltd., Lianyungang (CN); Shanghai Hengrui Pharmaceutical Co., Ltd., Shanghai (CN)

(72) Inventors: Hua Zhang, Shanghai (CN); Lianjun Shen, Shanghai (CN); Qing Su, Shanghai (CN); Weikang Tao, Shanghai (CN)

(21) Appl. No.: 16/967,294

(22) PCT Filed: Feb. 1, 2019
(86) PCT No.: PCT/CN2019/074392

§ 371 Date: Aug. 4, 2020

(30) Foreign Application Priority Data
Feb. 11, 2018 (CN) 201810140396.8

(51) Int. Cl. C07K 14/725; C12N 9/22; A61P 35/00; C12N 15/63; C12N 5/0783

(52) U.S. Cl. CPC C07K 14/7051; C12N 9/22; A61P 35/00; C12N 15/63; C12N 5/0636; C12N 2310/20; C07K 2319/02; C07K 2319/03

(57) ABSTRACT

An isolated CD19-binding chimeric antigen receptor, a CRISPR-Cas9 system-based method for knocking out TRAC, B2M and PD-1 genes in T cells in vitro, crRNA used in the method, gene knockout T cells obtained according to the method, and a use thereof.

### CROSS-REFERENCE TO RELATED APPLICATIONS

This application is a U.S. National Phase of International PCT Application No. PCT/CN2019/074392 filed Feb. 1, 2019, which claims priority to Chinese Patent Application Serial No. 201810140396.8 filed Feb. 11, 2018, the contents of each application are incorporated herein by reference in their entirety.

### SEQUENCE LISTING

This application incorporates by reference the material in the ASCII text file titled English_Translation_of_Sequence_Listing.txt, which was created on Feb. 23, 2021 and is 76.9 KB.

### FIELD OF THE INVENTION

The present disclosure belongs to the field of biomedicine. In particular, the present disclosure relates to a chimeric antigen receptor, cells comprising the chimeric antigen receptor, and uses thereof.

### BACKGROUND OF THE INVENTION

The statements herein merely provide background information related to the present invention and do not necessarily constitute the prior art.

Lymphatic malignancies, including lymphocytic leukemia and lymphoma, are tumors that occur on lymphocytes such as B cells, T cells, and NK cells. At present, there are many difficulties in the treatment, especially for the recurrent and refractory diseases that are often encountered in clinical practice. In the past 10 years, great progress has been made in the clinical treatment of lymphatic tumors. Anti-CD20 monoclonal antibodies have been widely used in CD20-positive B cell non-Hodgkin's lymphoma, and have achieved good curative effect. They have become clinical first-line medication. However, since CD20 is not expressed on the cells of some B lymphoma, acute and chronic B lymphoblastic leukemia, anti-CD20 antibodies such as rituximab have no obvious therapeutic effect on them. There is an urgent need for a new therapy to improve the cure rate of lymphoma and acute and chronic lymphocytic leukemia.

Chimeric antigen receptor T cells (CAR-T or CART) are T lymphocytes expressing specific CAR through genetic modification, and such cells can specifically recognize the target antigen and kill the target cells. CAR-T cells have high affinity to specific tumor antigen, and thereby can efficiently kill the tumor cells expressing the antigen. CD19 is specifically expressed on the surface of B lymphocytes at different stages of differentiation, and both B cell lymphoma and B lymphocyte leukemia express CD19 antigen. Therefore, construction of chimeric antigen receptor CART cells recognizing CD19 can achieve the purpose of effectively treating B lymphocytic tumors.

CD19-CART cells can recognize the specific CD19 target of B lymphocytic leukemia and attack the B lymphocytes expressing CD19 antigen by releasing cytokines such as perforin and granzyme, thereby promote to eliminate malignant lymphocytes from the organism. Sloan-Kettering Cancer Center in the United States has applied autologous 19-28zCAR-T technology in the treatment of refractory and relapsed acute B-cell lymphocytic leukemia (B-ALL), 14 of 16 patients achieved complete remission (CR), and the therapeutic effect was also found in Philadelphia chromosome-positive acute lymphocytic leukemia (Ph+ALL). Treatment with CART also creates prerequisites for allogeneic hematopoietic stem cell transplantation. The University of Pennsylvania also reported the results of the treatment of B-cell tumors with 19-CD137zCART, 27 out of 30 cases of refractory and relapsed B-ALL achieved CR, the rate of 6-month disease-free survival was 67%, and the overall survival rate reached 78%. Currently, Novartis, co-working with the University of Pennsylvania, received a marketing approval of the first CART cell as therapeutic drug for relapsed/refractory ALL in children, and thereafter Kite acquired a marketing approval of the second CAR-T as drug for non-Hodgkin's lymphoma.

Currently the approved CART products are produced by autologous cells, and have disadvantages of: long production cycle, high production cost, and qualified CAR-T cells cannot be produced in some patients. Such disadvantages make this technology incapable of being widely applied in patients. The TCR gene on the surface of T cells was knocked out in universal CAR-T cells (UCARTs), thereby eliminating or greatly reducing the GvHD efficiency. In addition, B2M-knockout can reduce the rejection of host to allogeneic cells. Meanwhile, allogeneic UCARTs are characterized in being ready-to-use and capable of being re-injected to the patients at a fixed dose, which avoid such a situation wherein the patients' T cells are unable to be expanded or prepared in time. Further, the large-scale preparation can reduce the production cost and is suitable for large-scale applications.

WO2014186585A2 and WO2016057821A2 patent application relate to a method for knocking out endogenous genes; WO2009091826, WO2012079000A1, WO2015187528, WO2015158671, WO2016014789, WO2016014576, WO2017049166 and WO2017173349 relate to the preparation and application of CAR-T cells; WO2015136001, WO2015140268, WO2015158671, WO2015193406 and WO2017032777 relate to the preparation and application of UCARTs. However, currently only the UCARTs from Cellectis SA, Pfizer Inc. and Shanghai BIORAY Inc. are at clinical research, Phase I stage, and there is no commercially available UCART cell as therapeutic drug. Therefore, there is a need to continuously explore new UCART cell as therapeutic drug.

### SUMMARY OF THE INVENTION

The purpose of the present disclosure is to overcome the problems in immunotherapy of the prior art, and to provide a genetically modified T cell comprising a nucleic acid encoding a chimeric antigen receptor that binds to CD19, and the endogenous genes TARC and B2M are knocked out in genetically modified T cell by CRISPR/Cas9 gene editing technology. Furthermore, the present disclosure also provides a crRNA with novel sequence for knocking out the endogenous genes TARC, B2M and PD-1, and provides use of gene knockout T cells obtained according to the methods of the present disclosure in the treatment or prevention of CD19-mediated diseases.

Some embodiments of the present disclosure provide a TCR- and PD-1- or B2M-double negative T cell and a method of constructing the same.

Further embodiments of the present disclosure provide a TCR-, B2M- and PD-1-triple negative T cell and a method of constructing the same.

Further, the above-mentioned TCR-negative T cells, TCR- and PD-1- or B2M-double-negative T cells and TCR/B2M/PD-1 triple negative T cells are sorted by magnetic beads, and are used for adoptive cell immunotherapy of tumors and the like.

In some embodiments, provided is a method of knocking out one or more target genes in T cells in vitro, the method comprises the steps of:

1) sgRNA(s) targeting one or more target genes in the T cells is/are contacted respectively with Cas9 protein to form protein RNA complex(s) (RNP(s));

2) the RNP(s) is/are mixed with oligo-deoxyribonucleic acid (N-oligo) or fish sperm DNA fragment; and used to transform the T cells, wherein the sgRNA(s) direct the Cas9 protein to a target sequence of the corresponding target gene and to hybridize with the target sequence, wherein the target gene is cleaved, and wherein the cleavage efficiency of the target gene is greater than 75%.

In some embodiments, the target gene is one or more or any combination selected from the group consisting of TRAC, TRBC, B2M and PD1 genes, and the sgRNA(s) target the coding sequence or the expression-regulating sequence of the target gene.

Further, the sgRNA(s) is/are formed by linking, from 5′ to 3′, a crRNA to a tracrRNA corresponding to the Cas9 protein, the crRNA targets the endogenous gene(s) and is 17 nt, 18 nt, 19 nt or 20 nt in length, preferably the crRNA is 17 nt in length.

In some embodiments, the oligodeoxyribonucleic acid is a double-stranded DNA with 100 bp, 250 bp, or any between 100 bp to 250 bp in length, or a single-stranded DNA with 100 nt, 250 nt, or any between 100 nt to 250 nt in length. Preferably, the sequence of the oligodeoxyribonucleic acid is shown in SEQ ID NO: 55.

In some embodiments, the crRNA(s) that target the TRAC gene is (are) any one or more crRNAs selected from the group consisting of SEQ ID NOs: 37, 38, 39, 40, 41, 42, 43, 44, 45, 46, 47 and 48, preferably the crRNA is shown in SEQ ID NO: 37; the sequence of crRNA that targets the B2M gene is shown in SEQ ID NO: 49; and the crRNA(s) that target the PD-1 gene is (are) any one or more selected from the group consisting of SEQ ID NOs: 50, 51 and 52, preferably the crRNA is shown in SEQ ID NO: 52.

In some embodiments, the Cas9 protein is Streptococcus Pyogenes-derived Cas9 protein, the amino acid sequence of which is shown in SEQ ID NO: 54, and the tracrRNA sequence corresponding to the Cas9 protein is shown in SEQ ID NO: 53.

In some embodiments, the T cell is selected from the group consisting of helper T cell, cytotoxic T cell, memory T cell, regulatory T cell, natural killer T cell, 76 T cell, CAR-T cell, and TCR-T cell.

In another aspect, the present disclosure also provides a T cell in which the target gene was knocked out, obtained according to the above method.

In another aspect, the disclosure also provides crRNAs for knocking out the TRAC gene, the crRNA targets the coding sequence or expression-regulating sequence of human TRAC gene and is selected from the group consisting of SEQ ID NOs: 37, 38, 39, 40, 41, 42, 43, 44, 45, 46, 47 and 48, preferably SEQ ID NO: 37.

In another aspect, the present disclosure also provides a crRNA for knocking out the B2M gene, the crRNA targets the coding sequence or expression-regulating sequence of human B2M gene and the sequence of the crRNA is shown in SEQ ID NO: 49.

In another aspect, the present disclosure also provides crRNAs for knocking out the PD-1 gene, the crRNA targets the coding sequence or expression-regulating sequence of human PD-1 gene and is selected from the group consisting of SEQ ID NOs: 50, 51 and 52, preferably SEQ ID NO: 52.

In another aspect, the present disclosure provides a kit for gene knockout, comprising sgRNA(s), Cas9 protein, and oligodeoxyribonucleic acid or fish sperm DNA, wherein the sgRNA(s) consist of the above-described one or more crRNAs separately linked to tracrRNA corresponding to the Cas9 protein.

In some embodiments of the kit for gene knockout, the oligodeoxyribonucleic acid is a double-stranded DNA with 100 bp, 250 bp, or any between 100 bp to 250 bp in length, or a single-stranded DNA with 100 nt, 250 nt, or any between 100 nt to 250 nt in length. Preferably, the sequence of the oligodeoxyribonucleic acid is shown in SEQ ID NO: 55.

In some embodiments of the kit for gene knockout, the Cas9 protein is Streptococcus Pyogenes-derived Cas9 protein, the amino acid sequence of which is shown in SEQ ID NO: 54, and the tracrRNA sequence corresponding to the Cas9 protein is shown in SEQ ID NO: 53.

In some embodiments, the disclosure provides use of the gene knockout T cells of the present disclosure in the preparation of an anti-tumor medicament.

In some embodiments, the present disclosure also provides use of the gene knockout T cells of the present disclosure in the preparation of a medicament for the prevention/treatment of infectious diseases caused by virus or bacteria.

In some embodiments, TCR, B2M or PD-1 are effectively knocked out using the designed crRNAs and method. The in vitro killing activity of TCR- and B2M- and/or PD-1-knockout CART cells is not affected by the gene knockout of TCR, B2M and/or PD-1.

The disclosure provides an isolated chimeric antigen receptor (CAR) comprising a CD19 antigen binding domain, a co-stimulatory signaling region, and a CD3ζ signaling domain, wherein the CD19 antigen binding domain comprises the amino acid sequence selected from the group consisting of SEQ ID NO: 18, SEQ ID NO: 20, SEQ ID NO: 22 and SEQ ID NO: 24, preferably comprises the amino acid sequence of SEQ ID NO: 20.

In some embodiments, the co-stimulatory signaling region comprises an intracellular domain of a co-stimulatory molecule selected from the group consisting of CD27, CD28, 4-1BB, OX40, CD30, CD40, PD-1, ICOS, lymphocyte function associated antigen-1 (LFA-1), CD2, CD7, LIGHT, NKG2C, B7-H3, and any combination thereof, preferably the 4-1BB co-stimulatory signaling region of SEQ ID NO: 12.

In some embodiments, the CD3ζ signaling domain comprises the amino acid sequence shown in SEQ ID NO: 14 or SEQ ID NO: 57.

In some embodiments, the CAR of the present disclosure further comprises an extracellular hinge domain, wherein the extracellular hinge domain comprises human CD8α leading signal region as shown in SEQ ID NO: 6 and human CD8α hinge region as shown in SEQ ID NO: 8.

In some embodiments, the CAR of the present disclosure further comprises CD8α transmembrane domain as shown in SEQ ID NO: 10.

In some embodiments, the CAR of the present disclosure comprises the amino acid sequence shown in SEQ ID NO:26, SEQ ID NO:28, SEQ ID NO:30 or SEQ ID NO:32, preferably SEQ ID NO:28.

The present disclosure further provides a series of nucleic acid molecules encoding the CARs as described above.

In some embodiments, the nucleic acid molecule comprises the nucleic acid sequence shown in SEQ ID NO: 17, SEQ ID NO: 19, SEQ ID NO: 21, or SEQ ID NO: 23.

In some embodiments, the nucleic acid molecule comprises a nucleic acid sequence encoding co-stimulatory signal transduction region and/or a nucleic acid sequence encoding CD3ζ signaling domain, preferably, the nucleic acid sequence encoding co-stimulatory signal transduction region is shown in SEQ ID NO:11, and the nucleic acid sequence encoding CD3ζ signaling domain is shown in SEQ ID NO: 13 or SEQ ID NO: 56.

In some embodiments, the nucleic acid molecule further comprises a nucleic acid sequence encoding extracellular hinge domain, preferably the nucleic acid sequence encoding extracellular hinge domain comprises human CD8α leading signal region shown in SEQ ID NO: 5 and human CD8α hinge region shown in SEQ ID NO: 7.

In some embodiments, the nucleic acid molecule further comprises a CD8α transmembrane domain shown in SEQ ID NO: 9.

In some embodiments, the nucleic acid molecule of the present disclosure encodes a CAR, wherein the CAR comprises the amino acid sequence shown in SEQ ID NO: 26, SEQ ID NO: 28, SEQ ID NO: 30, or SEQ ID NO: 32, preferably, SEQ ID NO:28.

In one embodiment, the nucleic acid molecule of the present disclosure encodes a CAR, wherein the nucleic acid molecule comprises the nucleic acid sequence shown in SEQ ID NO: 25, SEQ ID NO: 27, SEQ ID NO: 29 or SEQ ID NO: 31, preferably SEQ ID NO: 27.

The present disclosure further provides a vector comprising a nucleic acid sequence encoding the above CAR.

In some embodiments, the vector described in the present disclosure is selected from the group consisting of DNA, RNA, plasmid, lentiviral vector, adenoviral vector, and retroviral vector, preferably lentiviral vector.

In some embodiments, the vector of the present disclosure further comprises a promoter, preferably comprises EF-1 promoter shown in SEQ ID NO: 4.

The disclosure further provides some T cells comprising a nucleic acid sequence encoding CAR.

The present disclosure further provides a method for generating T cells comprising a nucleic acid sequence encoding CAR, the method comprises the step of introducing a nucleic acid encoding chimeric antigen receptor (CAR) into the T cells.

The present disclosure further provides some compositions comprising one or more selected from the group consisting of:

(i) an isolated CAR as described above,

(ii) a nucleic acid molecule encoding the CAR as described above,

(iii) a vector comprising the nucleic acid molecule encoding the CAR as described above, and

(iv) modified T cells comprising the CAR as described above.

The present disclosure further provides some modified T cells comprising:

a nucleic acid capable of down-regulating gene expression of an endogenous gene, the endogenous gene is one or more or any combination selected from the group consisting of TRAC, B2M and PD-1; and

a nucleic acid encoding chimeric antigen receptor (CAR), the CAR comprises a CD19 antigen binding domain, a co-stimulatory signaling region and a CD3ζ signaling domain, wherein the CD19 antigen binding domain comprises the amino acid sequence shown in SEQ ID NO: 18, SEQ ID NO: 20, SEQ ID NO: 22 or SEQ ID NO: 24.

In some embodiments of the modified T cells of the present disclosure, the nucleic acid capable of down-regulating the expression of an endogenous gene in T cell is selected from the group consisting of antisense RNA, antigomer RNA, siRNA, shRNA, and CRISPR-Cas9 systems, preferably CRISPR-Cas9 system.

In some embodiments, the Cas9 protein is a Cas9 from Streptococcus Pyogenes, the amino acid sequence of which is shown in SEQ ID NO:54, and the corresponding tracrRNA sequence is shown in SEQ ID NO:55.

In some embodiments, the CRISPR-Cas9 system further comprises a sgRNA that targets the coding sequence of an endogenous gene or the expression-regulation sequence of the endogenous gene, wherein the sgRNA is/are formed by linking, from 5′ to 3′, a crRNA targeting the endogenous gene(s) with 17 nt, 18 nt, 19 nt or 20 nt in length to a tracrRNA corresponding to the Cas9 protein.

In one embodiment of the modified T cell of the present disclosure, the endogenous gene is selected from the group consisting of TRAC and B2M.

In some embodiments of the modified T cells of the present disclosure, the crRNA that targets the endogenous gene TRAC is any one or more crRNAs selected from the group consisting of SEQ ID NOs: 37, 38, 39, 40, 41, 42, 43, 44, 45, 46, 47 and 48, preferably SEQ ID NO: 47; the crRNA that targets the endogenous gene B2M is shown in SEQ ID NO: 49; the crRNA that targets the endogenous gene PD-1 is shown in SEQ ID NO: 50, 51 or 52, preferably SEQ ID NO: 52.

In some embodiments of the modified T cells of the present disclosure, the co-stimulatory signaling region is a 4-1BB co-stimulatory signaling region, the amino acid sequence of which is shown in SEQ ID NO: 12.

In some embodiments of the modified T cells of the present disclosure, the CD3ζ signaling domain comprises the amino acid sequence shown in SEQ ID NO: 14.

In some embodiments of the modified T cells of the present disclosure, the CAR further comprises an extracellular hinge domain, wherein the extracellular hinge domain comprises the human CD8α leading signal region shown in SEQ ID NO:6 and the human CD8α hinge region shown in SEQ ID NO: 8.

In some embodiments of the modified T cells of the present disclosure, the CAR further comprises the CD8α transmembrane domain shown in SEQ ID NO: 10.

In some embodiments of the modified T cells of the present disclosure, the CAR comprises the amino acid sequence shown in SEQ ID NO: 26, SEQ ID NO: 28, SEQ ID NO: 30 or SEQ ID NO: 32, preferably, comprises the amino acid sequence shown in SEQ ID NO: 28.

The disclosure further provides some modified T cells, comprising:

nucleic acids capable of down-regulating gene expression of endogenous gene TRAC and B2M in the T cells, wherein the crRNA which down-regulates endogenous gene TRAC is shown in SEQ ID NOs: 37, 38, 39, 40, 41, 42, 43, 44, 45, 46, 47 or 48, preferably SEQ ID NO: 47, the crRNA which down-regulates the endogenous gene B2M is shown in SEQ ID NO: 49, and the crRNA that targets the endogenous gene PD-1 is shown in SEQ ID NO: 50, 51 or 52, preferably SEQ ID NO: 52; and

a nucleic acid encoding chimeric antigen receptor (CAR), wherein the CAR comprises the amino acid sequence shown in SEQ ID NO: 26, SEQ ID NO: 28, SEQ ID NO: 30 or SEQ ID NO: 32, preferably comprises the amino acid sequence shown in SEQ ID NO: 28.

The present disclosure further provides some modified T cells, comprising:

nucleic acids capable of down-regulating gene expression of endogenous gene TRAC and B2M in the T cells, wherein the crRNA which down-regulates endogenous gene TRAC in the T cells is shown in SEQ ID NO: 47, the crRNA which down-regulates endogenous gene B2M is shown in SEQ ID NO: 49, the crRNA that down-regulates endogenous gene PD-1 is shown in SEQ ID NO: 52; and the chimeric antigen receptor comprises the amino acid sequence shown in SEQ ID NO: 26, SEQ ID NO: 28, SEQ ID NO: 30 or SEQ ID NO: 32, preferably comprises the amino acid sequence shown in SEQ ID NO: 28; most preferably the modified T cells are UCART19^{TCR-/-} (single knockout: TCR knockout) or UCART19^{TCR-/-B2M-/-} (double knockout: TCR and B2M knockout) or UCART19^{TCR-/-B2M-/-PD-1-/-} (triple knockout: TCR, B2M and PD-1 knockout).

The present disclosure further provides a pharmaceutical composition comprising the above modified T cells.

The present disclosure further provides a method for preparing the above modified T cells, comprising:

(1) introducing a nucleic acid encoding chimeric antigen receptor (CAR) into the T cells;

(2) introducing a nucleic acid of sgRNA into the T cells by CRISPR-Cas9 system, said sgRNA is capable of down-regulating the expression of an endogenous target gene in the T cells, the endogenous target gene is selected from the group consisting of TARC and B2M.

In some embodiments of the method for preparing modified T cells of the present disclosure, the CAR comprises the amino acid sequence shown in SEQ ID NO:26, SEQ ID NO:28, SEQ ID NO:30 or SEQ ID NO:32.

In some embodiments of the method for preparing modified T cells of the present disclosure, the crRNA that targets the endogenous gene TRAC is any one or more or any combination selected from the group consisting of SEQ ID NOs: 37, 38, 39, 40, 41, 42, 43, 44, 45, 46, 47 and 48, preferably SEQ ID NO: 47; the crRNA that targets endogenous gene B2M is shown in SEQ ID NO: 49.

In some embodiments of the method for preparing modified T cells, the T cells are obtained from peripheral blood mononuclear cells, cord blood cells, purified T cell population or T cell lines.

In some embodiments of the method for preparing modified T cells of the present disclosure, the method further comprises expanding the T cells.

In some embodiments of the method for preparing modified T cells of the present disclosure, the step of expanding the T cell comprises stimulating the expanded T cell population with at least one molecule or cytokine selected from the group consisting of CD3, CD27, CD28, CD83, CD86, CD127, 4-1BBL, IL2, IL21, IL-15, IL-7, PD1-CD28 and PD-1.

In some embodiments, the method for preparing modified T cells of the disclosure further comprises cryopreserving the T cells.

In some embodiments, the method for preparing modified T cells of the present disclosure further comprises the step of thawing the cryopreserved T cells and then introducing the nucleic acid into the T cells.

In some embodiments of the method for preparing modified T cells of the present disclosure, wherein introduction of the nucleic acid is selected from the group consisting of transduction of the expanded T cells, transfection of the expanded T cells, and electroporation of the expanded T cells.

In some embodiments, the pharmaceutical composition of the present disclosure further comprises a pharmaceutically acceptable carrier, diluent or excipient.

In some embodiments, the pharmaceutical composition of the present disclosure further comprises a buffer.

In some embodiments of the pharmaceutical composition of the present disclosure, the buffer is neutral buffered saline or phosphate buffered saline.

In some embodiments, the pharmaceutical composition of the present disclosure further comprises an injectable freezing medium.

In some embodiments of the pharmaceutical composition of the present disclosure, the injectable freezing medium comprises plasmalyte-A, dextrose, NaCl, DMSO, dextran, and human serum albumin.

In some embodiments, the pharmaceutical composition of the present disclosure further comprises one or more cytokines.

The present disclosure further provides the use of the nucleic acid molecule encoding CAR, the vector comprising the nucleic acid molecule encoding CAR, the T cells comprising CAR, the T cells comprising nucleic acid(s) capable of down-regulating gene expression of the endogenous gene TRAC and B2M and a nucleic acid encoding CAR, or the use of the composition comprising the above components in the manufacture of a medicament for the treatment or prevention of a CD19-mediated disease.

The present disclosure further provides the nucleic acid molecule encoding CAR, the vector comprising the nucleic acid molecule encoding CAR, the T cells comprising CAR, the T cells comprising nucleic acid(s) capable of down-regulating gene expression of the endogenous gene TRAC and B2M and a nucleic acid encoding CAR, or the composition comprising the above components, for use in the treatment or prevention of a CD19-mediated disease.

The present disclosure further provides a method of treating or preventing a CD19-mediated disease, the method comprises administering to a subject an effective amount of the nucleic acid molecule encoding CAR, the vector comprising the nucleic acid molecule encoding CAR, the T cells comprising CAR, the T cells comprising nucleic acid(s) capable of down-regulating gene expression of the endogenous gene TRAC and B2M and a nucleic acid encoding CAR, or the composition comprising the above components.

In other embodiments, the above method comprises administering to the subject effect dose of cells which have been genetically modified to express CAR or effect dose of cells comprising nucleic acid(s) capable of down-regulating gene expression of the endogenous gene TRAC and B2M and a nucleic acid encoding CAR, wherein the CAR comprises the amino acid sequence shown in SEQ ID NO: 26, SEQ ID NO: 28, SEQ ID NO: 30 or SEQ ID NO: 32, preferably comprises the amino acid sequence shown in SEQ ID NO: 28, wherein the crRNA that down-regulates the endogenous gene TRAC is any one or more any combination selected from the group consisting of SEQ ID NO: 37, 37, 38, 39, 40, 41, 42, 43, 44, 45, 46, 47 and 48, preferably SEQ ID NO: 47; wherein the crRNA that down-regulates the endogenous gene B2M is shown in SEQ ID NO: 49, and wherein the crRNA that targets the endogenous gene PD-1 is anyone or more selected from the group consisting of SEQ ID NO: 50, 51 and 52, preferably SEQ ID NO: 52.

In one embodiment, the CD19 mediated disease is selected from the group consisting of cancer, infectious disease caused by virus or bacteria, and autoimmune disease, preferably cancer, more preferably breast cancer, prostate cancer, ovarian cancer, cervical cancer, skin cancer, pancreatic cancer, colorectal cancer, kidney cancer, liver cancer, brain cancer, hematological cancer, lung cancer, and thyroid cancer, most preferred hematological cancer.

In one embodiment, the hematological cancer is selected from the group consisting of leukemia, including acute leukemia, such as acute lymphocytic leukemia, acute myelocytic leukemia, acute myelogenous leukemia and myeloblastic leukemia, promyelocytic leukemia, myelomonocytic leukemia, monocytic leukemia and erythroleukemia; and chronic leukemia, such as chronic myelocytic (granulocytic) leukemia, chronic myelogenous leukemia and chronic lymphocytic leukemia and refractory CD19⁺ leukemia and lymphoma; polycythemia vera, lymphoma, mantle cell lymphoma, diffuse large B-cell lymphoma, Hodgkin's disease, non-Hodgkin's lymphoma, multiple myeloma, Waldenstrom's macroglobulinaemia, heavy chain disease, myelodysplastic syndrome, hairy cell leukemia and myelodysplasia; preferably acute lymphocytic leukemia or chronic lymphocytic leukemia.

### DESCRIPTION OF THE DRAWINGS

FIG. 1: Identification of the purity of the screened UCART19 cells.

FIG. 2: Transfection efficiency of lentivirus.

FIG. 3: Comparison of knockout efficiency of different delivery systems. The results show that RNP delivery method has the highest gene knockout efficiency in Jurkat cells.

FIG. 4A-4C: Effects of N-oligo and fish sperm DNA on gene knockout efficiency by CRISPR-Cas9 system in T cells. FIG. 4A shows comparison of gene knockout efficiency in T cells; FIG. 4B shows comparison of gene knockout efficiency in CAR-T cells; FIG. 4C shows the effect of fish sperm DNA fragments on gene knockout efficiency in T cells.

FIG. 5: Determination of B2M knockout efficiency in T cells. The results show that B2M knockout efficiency was up to 81.7%.

FIG. 6: Determination of PD-1 gene knockout efficiency in T cells. The results show that all the three crRNAs significantly knocked down PD-1.

FIG. 7A-7B: Analysis of gene mutations resulting from RNP and N-Oligo or fish sperm DNA. FIG. 7A shows analysis result for TRAC, and FIG. 7B shows analysis result for B2M.

FIGS. 8A-8C: Analysis of off-target rate with RNP. FIG. 8A shows the analysis results of the TRAC gene off-target rate; FIG. 8B shows the analysis results of the B2M gene off-target rate; and FIG. 8C shows analysis results of the PD-1 gene off-target rate.

FIG. 9A-9B: Analysis of CD25 and CD69 activation in TRAC knockout T cells. FIG. 9A shows comparison of CD69 activation; FIG. 9B shows comparison of CD25 activation.

FIG. 10: Killing effect of CART19 on CD19 positive cells K562-CD19 and K562.

FIG. 11: Killing effect of CART19 on Raji tumor cells.

FIG. 12A-12B: Cytokine release during killing of Raji and Daudi tumor cells by CART19-N2.

FIG. 13: Comparison of the killing ability of CART19 and UCART19 cells on tumor target cells. FIG. 13A shows the killing effect on Dudi cells; FIG. 13B shows the killing effect on Raji cells; and FIG. 13C shows the killing effect on Nalm6 cells.

FIG. 14A to 14C: The results of measurement of the expression level of cell surface CD107a in the process of in vitro killing target cells by CART19 and UCART19 cells. FIG. 14A shows the expression level of cell surface CD107a in the process of killing Daudi cells; FIG. 14B shows the expression level of cell surface CD107a in the process of killing Raji cells; and FIG. 14C shows the expression level of cell surface CD107a in the process of killing Nalm6 cells.

FIG. 15A-15D: Analysis of antitumor activity of CART19 cells in mouse in vivo. FIG. 15A shows a flow chart of establishment of NOG mouse model and injection of CART19 cells; FIG. 15B shows the results of pictures 5 weeks after injection; FIG. 15C shows the statistical results of bioluminescence intensity in mice; FIG. 15D shows the overall survival of CART19 cells in mouse.

FIG. 16A-16B: Analysis results of antitumor activity in mouse of K562 cells-stimulated CART19 and UCART19 cells. FIG. 16A shows the tumor burden in mouse after CART19 and UCART19 without or with secondary stimulation with K562-CD19 cells were injected into NOG mouse model; and FIG. 16B shows the survival rates of NOG mouse model injected with CART19 and UCART19 without or with secondary stimulation with K562-CD19 cells.

FIG. 17A-17B: Changes in the number of human T cells in peripheral blood of mice within 3 weeks after CART injection. FIG. 17A shows the proliferation profile of negative control CART-MSN cells in mice; and FIG. 17B shows the proliferation profile of CART19-N2 cells in mice.

FIG. 18A-18D: Survival rate, body weight change, and proliferation profile of human T cells in mice after injection of T-mock cells and T-TCR-cells. FIG. 18A shows the survival rate of mice after injection of different cells; FIG. 18B shows body weight changes of mice; FIG. 18C shows the proportion of CD45⁺ cells in mice after injection of CTL-019 cells; and FIG. 18D shows the proportion of CD45⁺ cells in mice after injection of CTL-019TCR^{-/-} cells.

### DETAILED DESCRIPTION OF THE DISCLOSURE

The present disclosure provides genetically modified UCART cells that can be used among different individuals, the genetically modified UCART cells have ability of specifically killing CD19 positive cells and tumor target cells in vitro and in vivo, and greatly reduce GvHD effects and allogeneic rejection.

### Terminology

In order to make the present disclosure more easily understood, certain technical and scientific terms are specifically defined below. Unless otherwise defined explicitly herein, all other technical and scientific terms used herein have the meaning commonly understood by one of ordinary skill in the art to which this disclosure belongs.

As used herein, the term “contacting” (i.e., contacting polynucleotide sequences with clustered regularly interspaced short palindromic repeats-related (Cas) protein and/or ribonucleic acids) is intended to include in vitro incubating the Cas protein and/or RNA, or ex vivo contact with cells. The step of contacting the polynucleotide sequences of the target genes with the Cas protein and/or ribonucleic acids disclosed herein can be carried out in any suitable manner. For example, the cells can be treated in the form of adherent or suspension culture. Cells contacted with the Cas protein and/or ribonucleic acids as disclosed herein can also be simultaneously or subsequently contacted with another agent, such as growth factor or other differentiation agent or environment, to stabilize or further differentiate the cells.

When applied to an isolated cell, the term “treating” includes subjecting the cell to any type of process or condition, or performing any type of operation or procedure on the cell. When applied to a subject, the term refers to providing the cell, in which the polynucleotide sequences of the target genes have been altered ex vivo according to the methods described herein, to an individual. The individual is typically ill or injured, or is at an increased risk of illness relative to the average member of the population and requires such attention, care or management.

The term “treating” as used herein, refers to administering to a subject an effective amount of cells in which the polynucleotide sequences of the target genes have been altered ex vivo according to the methods described herein, such that at least one symptom of the disease from which the subject suffered is alleviated or the disease is improved, for example, beneficial or desired clinical outcomes. For the purposes of the present disclosure, beneficial or desired clinical outcomes include, but are not limited to, alleviation of one or more symptoms, reduction of the disease level, stabilization of the disease state (i.e., no deterioration), delay or retardation in disease progression, improvement or mitigation of the disease state, and remission (whether partial or complete remission), whether detectable or undetectable. Treatment may mean prolonging survival as compared to the expected survival in the absence of treatment. Thus, those skilled in the art recognize that treatment may improve disease conditions, but may not be a complete cure for the disease. As used herein, the term “treatment” includes prophylaxis. Alternatively, treatment is “effective” in case that the progression of the disease is reduced or ceased. “Treatment” can also mean prolonging survival as compared to the expected survival in the absence of treatment. Patients in need of treatment include those who have been diagnosed having disease associated with the expression of polynucleotide sequences, and those who may develop such disease due to genetic susceptibility or other factors.

As used herein, “mutant cell” refers to a cell that differs from its original genotype. In some examples, “mutant cell” exhibits a mutant phenotype, for example, when a functionally normal gene is altered using the CRISPR/Cas system of the present disclosure. In other examples “mutant cell” exhibits a wild-type phenotype, for example, when a mutant genotype is corrected using the CRISPR/Cas system of the present disclosure. In some embodiments, the polynucleotide sequence of a target gene in a cell is altered to correct or repair the gene mutation (e.g., to restore the normal genotype of the cell). In some embodiments, the polynucleotide sequence of a target gene in a cell is altered to induce a genetic mutation (e.g., to disrupt the function of a gene or genomic element).

In some embodiments, the alteration is an indel. “Indel” as used herein refers to mutations resulting from insertions, deletions, or a combination thereof. As will be understood by those of skill in the art, an indel in a coding region of a genomic sequence will result in frameshift mutations unless the length of the indel is a multiple of three. In some embodiments, alteration is point mutation. “Point mutation” as used herein refers to a substitution that replaces one of the nucleotide. The CRISPR/Cas system of the present disclosure can be used to induce an indel of any length or a point mutations in a target polynucleotide sequences.

“Oligodeoxyribonucleic acid” or “N-oligo” refers to a deoxyribonucleic acid fragment with random sequence which is transformed into a cell together with RNP when gene knockout is performed with RNP delivery system. Preferably it refers to a double-stranded DNA with 100-250 bp in length, or a single-stranded DNA with 100-250 nt in length.

“Fish sperm DNA fragment” refer to small molecule fragment produced by mechanically shearing solution containing salmon sperm DNA. For example, 1% salmon sperm DNA solution is repeatedly aspirated with a 7-gauge needle to cut DNAs into small molecules, aliquoted and stored.

“Knockout” as used herein includes deleting all or part of target polynucleotide sequence in a way that interferes with the function of the target polynucleotides. For example, a knockout can be achieved by altering of a target polynucleotide sequence, and the alteration is performed by inducing indel in the target polynucleotide sequence in a functional domain (e.g., DNA binding domain). Based on the details described herein, one of skill in the art will readily understand how to use the CRISPR/Cas system of the present disclosure to knock out a target polynucleotide or a portion thereof.

In some embodiments, cleavage of the target gene results in decreased expression of the target gene. The term “decrease” is used herein generally to mean decreasing by a statistically significant amount. However, to avoid confusion, “decrease” means a decrease of at least 10% compared to a reference level, such as a decrease of at least about 20%, or at least about 30%, or at least about 40%, or at least about 50%, or at least about 60%, or at least about 70%, or at least about 75%, or at least about 80%, or at least about 90%, or a decrease of up to (and including) 100% compared to a reference level (i.e., a level corresponding to the absence of expression, when compared to the reference sample), or a decrease of any between 10% and 100%.

The term “statistically significant” or “significantly” refers to statistical significance and generally means a two standard deviations (2SD) less or below the normal marker concentration. The term means statistical evidence of the presence of difference. It is defined as the probability of making a decision to reject a hypothesis when the hypothesis is actually true. The decision is often expressed as p value.

In some embodiments, cleavage of a target gene is cleavage of a homozygous target gene. In some embodiments, cleavage of a target gene is cleavage of a heterozygous target gene.

The term “Cas9 protein” (also known as CRISPR-related endonuclease Cas9/Csnl) is a polypeptide comprising 1368 amino acids. An exemplary amino acid sequence of the Cas9 protein is shown in SEQ ID NO: 53. Cas9 contains two endonuclease domains, RuvC-like domain (residues 7-22, 759-766, and 982-989), which cleaves target DNA non-complementary to crRNA, and HNH nuclease domain (residues 810-872), which cleaves target DNA complementary to the crRNA.

The term “T cell receptor (TCR)” is a heterodimeric protein receptor for a specific antigenic peptide presented by the major histocompatibility complex (MHC). In the immune system, the binding of the antigen-specific TCR to the pMHC complex triggers direct physical contact between T cells and antigen presenting cells (APCs), and then T cells interact with other cell membrane surface molecules of APC, which causes a series of subsequent cell signaling and other physiological responses that allow different antigen-specific T cells to exert immune effects on their target cells.

TCR is a glycoprotein on the cell membrane surface in the form of a heterodimer formed by α chain/β chain or γ chain/δ chain. The TCR heterodimer in 95% of T cells consists of α and β chain, while 5% of T cells have TCR consisting of γ and δ chain. The native αβ heterodimeric TCR has α chain and β chain, and the α chain and the β chain constitute a subunit of the αβ heterodimeric TCR. Broadly, the α chain and the β chain comprise variable regions, a linker region and constant regions, and the R chain typically further contains a short diversified region between the variable region and the linker region, but this diversified region is often considered as a portion of the linker region. Each variable region comprises three CDRs (complementarity determining regions), CDR1, CDR2 and CDR3, which are interspersed in framework regions. The CDR regions determine the binding of the TCR to the pMHC complex, wherein the CDR3 is recombinantly composed of the variable regions and the linker region, and is referred to as the hypervariable region. It is generally considered that each of a and p chains of TCR has two “domains”, namely a variable domain and a constant domain, and the variable domain consists of variable region linked to the linker region. The sequence of the TCR constant domain can be found in the public database of the International Immunogenetics Information System (IMGT). For example, the constant domain sequence of the TCR molecule α chain is “TRAC*01”, and the constant domain sequence of the TCR molecule β chain is “TRBC1*01” or “TRBC2*01”. In addition, the α and β chains of TCR also contain transmembrane region and a very short cytoplasmic region.

“B2M”, also known as β-2 microglobulin, is the light chain of MHC class I molecules and is therefore an indispensable part of the major histocompatibility complex. In human, B2M is encoded by the b2m gene which is located on chromosome 15 and opposite to other MHC genes (as a cluster of genes) located on chromosome 6. The human protein consists of 119 amino acids and has a molecular weight of 11,800 Daltons. β-2 microglobulin deficient murine model has demonstrated that B2M is essential for MHC class I expression on cell surface and for stability of peptide binding cleft.

“PD-1” or “PD1” is a 50-55 kDa type I transmembrane receptor, which was originally identified in T cell line that underwent activation-induced apoptosis. PD-1 is expressed on T cells, B cells and macrophages. The PD-1 ligands are members of B7 family, PD-L1 (B7-H1) and PD-L2 (B7-DC).

PD-1 is a member of the immunoglobulin (Ig) superfamily and contains a single IgV-like domain in its extracellular region. The PD-1 cytoplasmic domain contains two tyrosine amino acids, of which the tyrosine much closer to the membrane (VAYEEL in mouse PD-1) is located within ITIM (an inhibitory motif of the immunoreceptor tyrosine). The presence of ITIM on PD-1 indicates that this molecule functions by recruiting cytosolic phosphatase to attenuate the signaling of antigen receptors. The human and murine PD-1 proteins share approximately 60% amino acid identity, with four conserved potential N-glycosylation sites and residues defining the Ig-V domain. The ITIM in cytoplasmic region and the ITIM-like motif around the carboxy terminal tyrosine (human and mouse TEYATI) are also conserved between human and murine orthologues.

The term “antibody” refers to an immunoglobulin molecule that specifically binds to an antigen. An antibody may be a complete immunoglobulin derived from natural source or derived from recombinant source, and may be an immunoreactive portion of a complete immunoglobulin. Antibodies are typically tetramers of immunoglobulin molecule. The antibodies of the present disclosure may exist in a variety of forms, including polyclonal antibodies, monoclonal antibodies, Fv, Fab, and F(ab)2, as well as single chain antibodies and humanized antibodies (Harlow et al, 1999, In: Using Antibodies: A Laboratory Manual, Cold Spring Harbor Laboratory Press, NY; Harlow et al, 1989, In: Antibodies: A Laboratory Manual, Cold Spring Harbor, N.Y.; Houston et al, 1988, Proc. Natl. Acad. Sci. USA 85: 5879-5883; Bird et al., 1988, Science 242: 423-426).

The term “antibody fragment” as used herein refers to a portion of an intact antibody and refers to the antigenic determinant variable region of an intact antibody. Examples of antibody fragments include, but are not limited to, Fab, Fab′, F(ab′)2 and Fv fragments, linear antibodies formed from antibody fragments, scFv antibodies, and multispecific antibodies.

The term “antibody heavy chain” as used herein refers to the larger chain of the two types of polypeptide chains present in all antibody molecules in their naturally occurring conformation.

The term “antibody light chain” as used herein refers to the smaller chain of the two types of polypeptide chains present in all antibody molecules in their naturally occurring conformation, and the κ and λ light chains are referred to as the two major isoforms of the antibody light chain.

The term “synthetic antibody” as used herein means an antibody produced by recombinant DNA techniques, such as, for example, an antibody expressed by a phage. The term should also be interpreted to mean an antibody that has been produced by synthesis mediated by synthetic DNA molecule (which encodes the antibody and the DNA molecule expresses the antibody protein or the amino acid sequence defining the antibody), wherein the DNA or amino acid sequence has been obtained by technology of synthesis of DNA or amino acid sequence available and well-known in the art.

The term “antigen” or “Ag” as used herein is defined as a molecule that elicits an immune response that can be involved in antibody production, or activation of specific immunocompetent cells. Those skilled in the art will appreciate that any macromolecule, including all proteins or peptides, can be used as an antigen. Furthermore, the antigen can be derived from recombinant or genomic DNA. One skilled in the art will appreciate any DNA, including nucleotide sequence or partial nucleotide sequence encoding a protein that elicits an immune response, encodes the term “antigen” as used herein. Furthermore, it will be understood by those skilled in the art that the antigen is not necessary to be individually encoded by full length nucleotide sequence of the gene. It will be readily apparent that the present disclosure includes, but is not limited to, use of partial nucleotide sequences of more than one gene, and these nucleotide sequences are arranged in different combinations to elicit a desired immune response. Furthermore, it will be understood by those skilled in the art that the antigen does not have to be encoded by a “gene” at all, and the antigen can be produced, synthesized or derived from a biological sample. Such biological samples can include, but are not limited to, tissue samples, tumor samples, cells, or biological fluids.

The term “autoantigen” means any autoantigen that is recognized by the immune system as foreign. Autoantigens include, but are not limited to, cellular proteins, phosphoproteins, cell surface proteins, cellular lipids, nucleic acids, glycoproteins, including cell surface receptors.

The term “chimeric antigen receptor” or “CAR” as used herein refers to an artificial T cell receptor engineered to be expressed on an immune effector cell and specifically bind to an antigen. CAR can be used as a therapy by adoptive cell transfer. T cells are removed from a patient and modified in a way that they can express a receptor specific for a particular antigen. CAR may also include an intracellular activation domain, a transmembrane domain, and an extracellular domain, including a tumor associated antigen binding region. In some aspects, the CAR comprises a single-chain variable fragment (scFv)-derived monoclonal antibody fused to a CD3-ζ transmembrane and intracellular domain. The designed specificity of the CAR can be derived from the ligand of the receptor (e.g., a peptide). In some embodiments, the CAR can target cancer by redirecting the specificity of T cells to express a CAR specific for a tumor associated antigen.

The term “anti-tumor effect” as used herein refers to a biological effect which may be clearly indicated by a decrease in tumor volume, a decrease in the number of tumor cells, a decrease in the number of metastases, an increase in life expectancy, or various physiological symptom associated with a cancerous condition. “Anti-tumor effect” can also be shown by the ability of the disclosed peptides, polynucleotides, cells and antibodies to prevent tumors.

The term “autoimmune disease” as used herein is defined as a disorder resulting from an autoimmune response. Autoimmune diseases are the result of inappropriate and excessive responses to autoantigens. Examples of autoimmune diseases include, but are not limited to, Addison's disease, alopecia areata, ankylosing spondylitis, autoimmune hepatitis, autoimmune mumps, Crohn's disease, diabetes (type 1), dystrophic bullous epidermis palliative, epididymitis, glomerulonephritis, Graves' disease, Guillain-Barre syndrome, Hashimoto's disease, hemolytic anemia, systemic lupus erythematosus, multiple sclerosis, myasthenia gravis, Pemphigus vulgaris, psoriasis, rheumatic fever, rheumatoid arthritis, sarcoidosis, scleroderma, Sjogren's syndrome, spondyloarthropathy, thyroiditis, vasculitis, vitiligo, myxedema, malignancy anemia, ulcerative colitis, etc.

The term “co-stimulatory ligand” as used herein includes a molecule on antigen presenting cells (e.g., APCs, dendritic cells, B cells, etc.) that specifically binds to an associated co-stimulatory molecule on T cell, thereby said co-stimulatory ligand provides, not only the primary signal (by binding the TCR/CD3 complex to the peptide-loaded MHC molecule), but also a signal that mediates T cell response, and said T cell response includes but not limited to proliferation, activation, differentiation, and the like. Co-stimulatory ligands can include, but are not limited to, CD7, B7-1 (CD80), B7-2 (CD86), PD-L1, PD-L2, 4-1BBL, OX40L, inducible co-stimulatory ligands (ICOS-L), intercellular adhesion molecule (ICAM), CD30L, CD40, CD70, CD83, HLA-G, MICA, MICB, HVEM, lymphotoxin β receptor, 3/TR6, ILT3, ILT4, HVEM, agonists or antibodies of Toll ligand-binding receptor and ligands specifically binding to B7-H3. Co-stimulatory ligands also include, inter alia, antibodies that specifically bind to co-stimulatory molecules present on T cells, said co-stimulatory molecules including but not limited to CD27, CD28, 4-1BB, OX40, CD30, CD40, PD-1, ICOS, lymphocyte function associated antigen-1 (LFA-1), CD2, CD7, LIGHT, NKG2C, B7-H3 and ligands that specifically bind to CD83.

“Co-stimulatory molecule” refers to an associated binding partner on T cells that specifically binds to a co-stimulatory ligand, thereby mediating co-stimulatory response of T cells, such as, but not limited to, proliferation. The co-stimulatory molecules include but are not limited to MHC Class I molecules, BTLA and Toll ligand receptors.

As used herein, “co-stimulatory signal” refers to a signal that associates with a primary signal, such as TCR/CD3 binding, and results in T cell proliferation and/or up- or down-regulation of key molecules.

As used herein, the term “autologous” refers to any substance derived from the same individual, which is subsequently reintroduced into the individual.

“Allogeneic” refers to a graft derived from a different animal of the same species.

“Xenogeneic” refers to a graft derived from an animal of a different species.

The term “cleavage” refers to breakage of a covalent bond, for example, in the backbone of a nucleic acid molecule. Cleavage can be initiated by a variety of methods including, but are not limited to, enzymatic cleavage or chemical hydrolysis of phosphodiester bonds. Cleavage is possible for both single-strand and double-strand. A double-strand may be cleaved by cleavage events of the two different single-strands. DNA cleavage can result in blunt ends or staggered ends. In certain embodiments, a fusion polypeptide can be used to target a cleaved double stranded DNA.

The term “CRISPR/CAS”, “clustered regular interspaced short palindromic repeats system” or “CRISPR” refers to a DNA locus comprising a short repeat of base sequence. Each repeat is followed by a short segment of spacer DNA that was previously exposed to virus. Bacteria and archaea have evolved an adaptive immune defense known as CRISPR-CRISPR-associated (Cas) system, which uses short RNA to direct the degradation of exogenous nucleic acids. In bacteria, the CRISPR system provides acquired immunity against invading foreign DNAs via RNA-guided DNA cleavage.

In Type II CRISPR/Cas system, “spacer region”, a short segment of exogenous DNA, is integrated into the CRISPR genomic locus and transcribed and processed into short CRISPR RNA (crRNA). These crRNAs were annealed with trans-activated crRNAs (tracrRNAs) and pathogenic DNA was sequence-specifically cleaved and silenced under the direction of Cas protein. Recent work has shown that a “seed” sequence within the crRNA and Protospacer Adjacent Motif (PAM) sequence containing a conserved dinucleotide upstream of the crRNA-binding region are required for Cas9 protein to recognize the target.

In order to direct Cas9 to cleave the sequence of interest, crRNA-tracrRNA fusion transcript can be designed based on human U6 polymerase III promoter, hereinafter referred to as “guide RNA” or “sgRNA”. CRISPR/CAS-mediated genome editing and regulation highlights its transformative potential for basic science, cell engineering and therapy.

The term “CRISPRi” refers to a CRISPR system for sequence-specifically repressing or inhibiting gene repression, such as at the transcriptional level.

As used herein, the term “exogenous” refers to any substance introduced into an organism, cell, tissue or system, or produced outside of the organism, cell, tissue or system.

As used herein, “endogenous” or “endogenic” refers to any substance derived from an organism, cell, tissue or system, or produced within the organism, cell, tissue or system.

The term “downregulation” as used herein refers to the reduction or elimination of gene expression of one or more genes.

The term “expansion” as used herein refers to an increase in the number, such as an increase in the number of T cells. In one embodiment, the number of ex vivo expanded T cells is increased relative to the number originally present in the culture. In another embodiment, the number of ex vivo expanded T cells is increased relative to the number of other cell types in the culture.

The term “ex vivo” as used herein refers to cells which have been removed from a living organism (e.g., human) and are propagated outside the organism (e.g., in a culture dish, test tube, or bioreactor).

The term “expression” as used herein is defined as the transcription and/or translation of particular nucleotide sequence driven by its promoter.

A “vector” is a composition of matter, it includes an isolated nucleic acid, and can be used to deliver the isolated nucleic acid into the cell. Numerous vectors are known in the art including, but not limited to, linear polynucleotides, polynucleotides associated with ionic or amphiphilic compounds, plasmids, and viruses. Thus, the term “vector” includes autonomously replicating plasmids or viruses. The term should also be interpreted to include non-plasmid and non-viral compounds that facilitate the transfer of nucleic acids into cells, such as, for example, polylysine compounds, liposomes, and the like. Examples of viral vectors include, but are not limited to, Sendai viral vectors, adenoviral vectors, adeno-associated viral vectors, retroviral vectors, lentiviral vectors, and the like.

“Expression vector” refers to a vector comprising a recombinant polynucleotide comprising an expression control sequence operably linked to a nucleotide sequence to be expressed. Expression vectors include sufficient cis-acting elements for expression; other elements for expression can be supplied by host cells or supplied in an in vitro expression system. Expression vectors include all those incorporating recombinant polynucleotides known in the art, such as cosmids, plasmids (e.g., naked or contained in liposomes), and viruses (e.g., Sendai virus, lentivirus, retrovirus, adenovirus and adeno-associated virus).

As used herein, “homologous” refers to the sequence identity of the subunits between two polymer molecules, for example, two nucleic acid molecules, such as two DNA molecules or two RNA molecules, or two polypeptide molecules. When the subunit positions in both molecules are occupied by the same monomeric subunit, for example, if the position in each of the two DNA molecules is occupied by adenine, they are homologous at that position. The homology between two sequences is a direct function of the number of matching or homologous positions; for example, if half positions in the two sequences (for example, five positions in a polymer with ten subunits in length) are homologous, the two sequences are 50% homologous; if 90% of the positions (e.g., 9 out of 10) are matched or homologous, the two sequences are 90% homologous.

As used herein, “identity” refers to the sequence identity of the subunit between two polymer molecules, particularly between two amino acid molecules, for example, between two polypeptide molecules. When two amino acid sequences share the same residue at the same position, for example, if the position in each of the two polypeptide molecules is occupied by arginine, they are identical at that position. In alignment, the identity or extent of two amino acid sequences having the same residue at the same position is often expressed as a percentage. The identity between two sequences is a direct function of the number of matching or homologous positions; for example, if half positions in the two sequences (for example, five positions in a polymer with ten amino acids in length) are homologous, the two sequences are 50% identical; if 90% of the positions (e.g., 9 out of 10) are matched or homologous, the two sequences are 90% identical.

The term “immunoglobulin” or “Ig” as used herein is defined as a class of proteins that function as antibodies. Antibodies expressed by B cells are sometimes referred to as BCR (B cell receptor) or antigen receptor. The five members included in such proteins are IgA, IgG, IgM, IgD, and IgE. IgA is a primary antibody present in body secretion such as saliva, tears, milk, gastrointestinal secretions, and mucus secretions of the respiratory and genitourinary tract. IgG is the most common circulating antibody. IgM is the major immunoglobulin produced in the primary immune response in most subjects. It is the most effective immunoglobulin in agglutination, complement binding and other antibody responses and is important in defensing bacteria and viruses. IgD is an immunoglobulin that does not have the well-known antibody function, but it can act as an antigen receptor. IgE is an immunoglobulin that mediates rapid allergic reaction by initiating release of mediators from mast cells and basophilic granulocyte, upon exposure to allergens.

The term “immune response” as used herein is defined as a cellular response to an antigen that occurs when lymphocytes recognize an antigen molecule as a foreigner and induce the formation of an antibody and/or the lymphocytes are activated to remove the antigen.

“Isolated” means being altered or removed from natural state. For example, a nucleic acid or peptide naturally present in a living animal is not “isolated”, but the same nucleic acid or peptide that is partially or completely separated from the coexisting material in its natural state is deemed as “isolated.” The isolated nucleic acid or protein may be present in substantially purified form, or may be present in a non-native environment, such as, for example, in host cells.

The term “knockdown” as used herein refers to a decrease in gene expression of one or more genes.

The term “knockout” as used herein refers to ablation of gene expression of one or more genes.

“Lentivirus” as used herein refers to a genus of the retroviridae. In retroviruses, lentivirus is the only virus that is capable of infecting non-dividing cells, such as HIV, S1V and FIV; They can transfer significant amount of genetic information into the DNA of host cells, hence they are the most efficient means of gene-delivery vectors. Vectors derived from lentivirus provide a means to accomplish significant levels of gene transfer in vivo.

The term “modified” as used herein means an altered state or structure of a molecule or cell of the present disclosure. Molecules can be modified in various ways, including chemical, structural and functional modification. Cells can be modified by the introduction of nucleic acids.

The term “regulation” as used herein means to mediate a detectable increase or decrease of the response level in a subject, compared to the response level in a subject without the treatment or not administrated with a compound, and/or compared to the response level in a subject without treatment but comparable in other aspects. The term includes disturbing and/or affecting a natural signal or response, thereby mediating a beneficial therapeutic response in a subject, preferably a human.

Unless otherwise specified, “nucleotide sequences encoding an amino acid sequence” include all nucleotide sequences which encode the same amino acid sequence, due to the degeneracy. The phrase “nucleotide sequences encoding a protein or RNA” may also include intron(s), to an extent that the nucleotide sequence encoding the protein may comprise (one or more) intron(s) in particular form.

The term “operably linked” refers to a functional linkage between a regulatory sequence and a heterologous nucleic acid sequence, which results in expression of the heterologous nucleic acid sequence. For example, when the first nucleic acid sequence is in a functional relationship with the second nucleic acid sequence, the first nucleic acid sequence is operably linked to the second nucleic acid sequence. For example, a promoter is operably linked to a coding sequence if the promoter affects the transcription or expression of the coding sequence. Typically, operably linked DNA sequence is adjacent to each other and, where necessary, two protein coding regions are ligated in the same reading frame.

The term “overexpressed” tumor antigen or “overexpression” of tumor antigen is intended to indicate abnormal expression level of a tumor antigen from a diseased region (such as cells from a solid tumor in a particular tissue or organ of a patient), relative to the expression level of a normal cell from the tissue or organ. Patients with solid tumors or hematological malignancies characterized by overexpressed tumor antigen can be determined by standard assays known in the art.

As used herein, the terms “peptide”, “polypeptide” and “protein” are used interchangeably and refer to a compound consisting of amino acid residues covalently linked by peptide bonds. The protein or peptide must contain at least two amino acids, and there is no limitation to the maximum number of amino acids constituting a protein or peptide sequence. Polypeptide include any peptide or protein comprising two or more amino acids connected to each other by peptide bond. As used herein, the term refers to both short chains (which are also commonly referred to in the art as, for example, peptide, oligopeptide, and oligomer); and longer chains (which are commonly referred to in the art as proteins), which has many types. “Polypeptide” includes, for example, biologically active fragment, substantially homologous polypeptide, oligopeptide, homodimer, heterodimer, variant of polypeptide, modified polypeptide, derivative, analog, fusion protein, and the like. Polypeptide includes natural peptide, recombinant peptide, synthetic peptide, or a combination thereof.

The term “promoter” as used herein is defined as a DNA sequence, which is necessary for initiating the specific transcription of a polynucleotide sequence, and is recognized by synthetic machine of a cell, or is introduced by synthetic machinery.

As used herein, the term “promoter/regulatory sequence” means a nucleic acid sequence required for expression of a gene product operably linked to the promoter/regulatory sequence. In some examples, the sequence may be a core promoter sequence, and in other examples, the sequence may also include enhancer sequence and other regulatory elements required for expression of the gene product. For example, a promoter/regulatory sequence can be a sequence that expresses a gene product in a tissue-specific manner.

“Signaling pathway” refers to the biochemical relationship among a variety of signaling molecules which function in transferring a signal from one part of a cell to another part of the cell.

“Cell surface receptors” include complexes of a molecule with other molecule(s), and they are capable of receiving and transmitting signals across the plasma membrane of a cell.

The term “specifically binds to” as used herein with respect to an antibody means that the antibody recognizes a specific antigen but does not substantially recognize or bind to other molecules in the sample. For example, an antibody that specifically binds to an antigen from one species can also bind to an antigen from one or more species. However, such cross-species reactivity per se does not change the specificity of class of the antibody. In some cases, an antibody that specifically binds to an antigen can also bind to an antigen of a different allelic form. However, such cross-reactivity per se does not change the specificity of the class of the antibody. In some instances, the terms “specifically bind to” or “specifically bind” may refer to the interaction of an antibody, protein or peptide with a second chemical species, meaning that the interaction is dependent on the presence of a particular structure of the chemical species (e.g., an antigen determinant or epitope); for example, an antibody recognizes and binds to a particular protein structure, rather than generally recognizes and binds to the protein. If an antibody is specific for epitope “A”, the presence of a molecule comprising epitope A (or free unlabeled A) in the reaction comprising the labeled “A” and the antibody will reduce the amount of labeled A bound to the antibody.

“Single-chain antibody” refers to an antibody formed by recombinant DNA techniques in which immunoglobulin heavy and light chain fragments are linked to the Fv region via an engineered span of amino acids. A variety of methods for generating single-chain antibodies are known, and are included in U.S. Pat. No. 4,694,778; Bird (1988) Science 242: 423-442; Huston et al. (1988) Proc. Natl. Acad. Sci. USA 85: 5879-5883; Ward et al (1989) Nature 334: 54454; Skerra et al (1988) Science 242: 1038-1041.

The term “stimulation” means mediating signal transduction event by binding to a stimulatory molecule (e.g., TCR/CD3 complex) and its associated ligand, for example, but not limited to, the first response induced by signal transduction via the TCR/CD3 complex. Stimulation can mediate altered expression of certain molecules, for example, down-regulation of TGF-β, and/or recombination of cytoskeletal structures.

“Stimulatory molecule”, as a term used herein, means a molecule on T cells that specifically binds to an associated stimulatory ligand present on an antigen presenting cell.

As used herein, “stimulatory ligand” refers to the following ligand: when present on antigen presenting cells (e.g., aAPC, dendritic cells, B-cells, etc.), it can specifically bind to the associated binding partner on T cells (referred to as “stimulatory molecule” herein), thereby mediates the primary response of T cells, including but not limited to activation, initiation of an immune response, proliferation, and the like.

Stimulatory ligands are well known in the art and include, inter alia, MHC class I molecules: loaded with peptide, anti-CD3 antibody, super-agonist anti-CD28 antibody and super-agonist anti-CD2 antibody.

The term “subject” is intended to include living organisms (e.g., mammals) in which an immune response can be elicited. The “subject” or “patient” as used therein may be human or non-human mammals. Non-human mammals include, for example, domestic animals and pets, such as sheep, bovine, porcine, canine, feline, and murine. Preferably, the subject is human.

“Substantially purified” cells as used herein are cells that are substantially free of other cell types. Substantially purified cells also refers to the cells that have been separated from other cell types which are normally associated with the cells in their naturally occurring state. In some cases, a substantially purified population of cells refers to a homogeneous population of cells. In other instances, the term simply refers to cells that have been separated from other cells that are normally associated with the cells in their native state. In some embodiments, the cells are cultured in vitro. In other embodiments, the cells are not cultured in vitro.

“Target site” or “target sequence” refers to genomic nucleic acid sequences, which define a portion of nucleic acid that can specifically bind to a binding molecule under conditions sufficiently for binding.

The term “therapeutic” as used herein means treatment and/or prevention.

Therapeutic effects are obtained by suppression, alleviation or eradication of the disease state.

The term “transfected” or “transformed” or “transduced” as used herein refers to a process by which an exogenous nucleic acid is transferred or introduced into a host cell. “Transfected” or “transformed” or “transduced” cells are those which have been transfected, transformed or transduced with exogenous nucleic acid(s). Cells include primary subject cells and their progeny.

The phrase “under transcriptional control” or “operably linked to” as used herein means that the promoter is in the correct position and orientation relative to the polynucleotide, thereby it can control the initiation of transcription by RNA polymerase and the expression of the polynucleotide.

The term “effective amount” or “therapeutically effective amount” refers to an amount of a subject compound that will elicit a biological or medical response in a tissue, system or subject which a researcher, veterinarian, doctor or other clinician is looking for.

The term “therapeutically effective amount” includes an amount of a compound which, when administered, is sufficient to prevent the development of one or more of the signs or symptoms of the disorder or disease, or to some extent alleviate one or more of the signs or symptoms of the disorder or disease being treated. The therapeutically effective amount will vary depending on the compound, the disease and its severity, and the age, weight of the subject to be treated, etc.

### DETAILED DESCRIPTION OF THE DISCLOSURE

### Examples

The present disclosure is further illustrated in detail by the following examples. These examples are for illustrative purposes only and are not intended to limit the scope of the disclosure.

In the examples of the present disclosure, where specific conditions are not described, the experiments are generally conducted under conventional conditions; or under conditions proposed by the material or product manufacturers. Where the source of the reagents is not specifically given, the reagents are the commercially available conventional reagents.

### Example 1. Construction of pLVX-EF1-CD19 CAR Lentiviral Vector

(1) Gene sequence information of human CD8α leading signal region (SEQ ID NO: 5), human CD8α hinge region (SEQ ID NO: 7), human CD8α transmembrane region (SEQ ID NO: 9), human 4-1BB intracellular region (SEQ ID NO: 11) and human CD3 intracellular region (SEQ ID NO: 13) was obtained from the NCBI website database; CD19scFv was derived from FMC63 antibody (See Mol Immunol. 1997; 34: 1157-1165.), and the nucleic acid sequences thereof are shown in CD19-N1 scFv (SEQ ID NO: 17), CD19-N2 scFv (SEQ ID NO: 19), CD19-N3 scFv (SEQ ID NO: 21) and CD19-N4 scFv (SEQ ID NO: 23), respectively.

(2) The nucleotides with the above sequences were synthesized by Nanjing Genscript Biotech Co., Ltd., and the restriction enzyme sites were added to both ends to obtain a complete CD19-CAR gene sequence CD19 CAR-N1 (SEQ ID NO: 25), CD19 CAR-N2 (SEQ ID NO: 27), CD19 CAR-N3 (SEQ ID NO: 29), CD19 CAR-N4 (SEQ ID NO: 31). The CD19-CAR structure, from 5′ to 3′, successively consists of CD19scFv, hinge structure, transmembrane structure, 4-1BB and CD3ζ.

(3) pLVX-EF1-MSC plasmid obtained

The CMV promoter was excised from pLVX-CMV-MCS (Clontech, pLVX-IRES-ZsGreen1, Cat. No. 632187) and the EF1 promoter (SEQ ID NO: 4) was excised from pCDH-EF1-MCS (purchased from System Biosciences, Cat. No. CD530A-2) with ClaI and EcoRI endonucleases. The pLVX-CMV-MCS vector from which CMV has been excised and the EF1 promoter fragment from pCDH-EF1-MCS were recovered by agarose gel electrophoresis. The EF1 promoter fragment was ligated into the vector pLVX-MCS by using the DNA Ligation Kit (Takara) to obtain a plasmid, pLVX-EF1-MSC, which was then transformed into competent E. coli TOP10. The plasmid was extracted and verified by sequencing to obtain the correct pLVX-EF1-MCS lentiviral vector. The sequencing primers were: PLVX-PF (SEQ ID NO: 1) and PCDHI-R (SEQ ID NO: 2). The sequence is shown below:

PLVX-PF:
(SEQ ID NO: 1)
CATTCGATTAGTGAACGGATCT
PCDHI-R:
(SEQ ID NO: 2)
GACGGCAATATGGTGGAA.

(4) The CD19-CAR nucleic acid molecule was digested with EcoRI (NEB) and NotI (NEB), and ligated into the lentiviral vector pLVX-EF1-MCS between EcoRI and NotI sites by DNA Ligation Kit (Takara) to obtain pLVX-EF1-CD19 CAR lentiviral vector: pLVX-EF1-002A (with CD19 CAR sequence of CD19 CAR-N1), CD19-CAR lentiviral vector pLVX-EF1-002B (with CD19 CAR sequence of CD19 CAR-N2), CD19-CAR lentiviral vector pLVX-EF1-002C (with CD19 CAR sequence is CD19 CAR-N3), CD19-CAR lentiviral vector pLVX-EF1-002D (the CD19 CAR sequence of CD19 CAR-N4), and then transformed into the competent E. coli TOP10. The resulting pLVX-EF1-CD19 CAR lentiviral vector was sequenced, and the sequencing primers were: pLVX-PF (SEQ ID NO: 1) and Xd-SR (SEQ ID NO: 3), and the sequences are as follows:

pLVX-PF:
(SEQ ID NO: 1)
CATTCGATTAGTGAACGGATCT
Xd-SR:
(SEQ ID NO: 3)
AAAGCCATACGGGAAGCAATA.

The monoclonal colonies with correct sequences were selected for activation and inoculation, and the lentiviral vector was extracted using QIAGEN EndoFree Plasmid Extraction Kit.

The relevant sequences involved in Example 1 are as follows:

EF1 promoter:
(SEQ ID NO: 4)
GGATCTGCGATCGCTCCGGTGCCCGTCAGTGGGCAGAGCGCACATCGCCCAC
AGTCCCCGAGAAGTTGGGGGGAGGGGTCGGCAATTGAACGGGTGCCTAGAGA
AGGTGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACGTTC
TTTTTCGCAACGGGTTTGCCGCCAGAACACAGCTGAAGCTTCGAGGGGCTCG
CATCTCTCCTTCACGCGCCCGCCGCCCTACCTGAGGCCGCCATCCACGCCGGT
TGAGTCGCGTTCTGCCGCCTCCCGCCTGTGGTGCCTCCTGAACTGCGTCCGCC
GTCTAGGTAAGTTTAAAGCTCAGGTCGAGACCGGGCCTTTGTCCGGCGCTCCC
TTGGAGCCTACCTAGACTCAGCCGGCTCTCCACGCTTTGCCTGACCCTGCTTG
CTCAACTCTACGTCTTTGTTTCGTTTTCTGTTCTGCGCCGTTACAGATCCAAGC
TGTGACCGGCGCCTAC
Nucleotide sequence of human CD8α leading signal region:
(SEQ ID NO: 5)
ATGGCCTTACCAGTGACCGCCTTGCTCCTGCCGCTGGCCTTGCTGCTCCACGC
CGCCAGGCCG
Amino acid sequence of human CD8α leading signal region:
(SEQ ID NO: 6)
MALPVTALLLPLALLLHAARP
Nucleotide sequence of human CD8α hinge region:
(SEQ ID NO: 7)
ACCACGACGCCAGCGCCGCGACCACCAACACCGGCGCCCACCATCGCGTCGC
AGCCCCTGTCCCTGCGCCCAGAGGCGTGCCGGCCAGCGGCGGGGGGCGCAGT
GCACACGAGGGGGCTGGACTTCGCCTGTGAT
Amino acid sequence of human CD8α hinge region:
(SEQ ID NO: 8)
TTTPAPRPPTPAPTIASQPLSLRPEACRPAAGGAVHTRGLDFACD
Nucleotide sequence of human CD8α transmembrane region:
(SEQ ID NO: 9)
ATCTACATCTGGGCGCCCTTGGCCGGGACTTGTGGGGTCCTTCTCCTGTCACTG
GTTATCACCCTTTACTGC
Amino acid sequence of human CD8α transmembrane region:
(SEQ ID NO: 10)
IYIWAPLAGTCGVLLLSLVITLYC
Nucleotide sequence of human 4-1BB intracellular region:
(SEQ ID NO: 11)
AAACGGGGCAGAAAGAAACTCCTGTATATATTCAAACAACCATTTATGAGACC
AGTACAAACTACTCAAGAGGAAGATGGCTGTAGCTGCCGATTTCCAGAAGAA
GAAGAAGGAGGATGTGAACTG
Amino acid sequence of human 4-1BB intracellular region:
(SEQ ID NO: 12)
KRGRKKLLYIFKQPFMRPVQTTQEEDGCSCRFPEEEEGGCEL
Nucleotide sequence of human CD3ζ intracellular region:
(SEQ ID NO: 13)
AGAGTGAAGTTCAGCAGGAGCGCAGACGCCCCCGCGTACAAGCAGGGCCAG
AACCAGCTCTATAACGAGCTCAATCTAGGACGAAGAGAGGAGTACGATGTTTT
GGACAAGAGACGTGGCCGGGACCCTGAGATGGGGGGAAAGCCGAGAAGGAA
GAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCT
TCACATGCAGGCCCTGCCCCCTCGC
Amino acid sequence of human CD3ζ intracellular region:
(SEQ ID NO: 14)
RVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHM
QALPPR
Heavy chain variable region amino acid sequence of FMC63 antibody:
(SEQ ID NO: 15)
EVKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDY
WGQGTSVTVSS
Light chain variable region amino acid sequence of FMC63 antibody:
(SEQ ID NO: 16)
DIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITRADAA
PTVSIFPP
Nucleotide sequence of CD19-N1 scFv:
(SEQ ID NO: 17)
GACATCCAGATGACACAGACTACATCCTCCCTGTCTGCCTCTCTGGGAGACAG
AGTCACCATCAGTTGCAGGGCAAGTCAGGACATTAGTAAATATTTAAATTGGTA
TCAGCAGAAACCAGATGGAACTGTTAAACTCCTGATCTACCATACATCAAGATT
ACACTCAGGAGTCCCATCAAGGTTCAGTGGCAGTGGGTCTGGAACAGATTATT
CTCTCACCATTAGCAACCTGGAGCAAGAAGATATTGCCACTTACTTTTGCCAAC
AGGGTAATACGCTTCCGTACACGTTCGGAGGGGGGACTAAGTTGGAAATAACA
GGTGGCGGTGGCTCGGGCGGTGGTGGGTCGGGTGGCGGCGGATCTGAGGTGA
AACTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGATTCG
CCAGCCTCCACGAAAGGGTCTGGAGTGGCTGGGAGTAATATGGGGTAGTGAA
ACCACATACTATAATTCAGCTCTCAAATCCAGACTGACCATCATCAAGGACAAC
TCCAAGAGCCAAGTTTTCTTAAAAATGAACAGTCTGCAAACTGATGACACAGC
CATTTACTACTGTGCCAAACATTATTACTACGGTGGTAGCTATGCTATGGACTAC
TGGGGCCAAGGAACCTCAGTCACCGTCTCCTCA
Amino acid sequence of CD19-N1 scFv:
(SEQ ID NO: 18)
DIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITGGGGS
GGGGSGGGGSEVKLQESGPGLVAPSQSLSVTCTVSGVSLPDYGVSWIRQPPRKGL
EWLGVIWGSETTYYNSALKSRLTIIKDNSKSQVFLKMNSLQTDDTAIYYCAKHYY
YGGSYAMDYWGQGTSVTVSS
Nucleotide sequence of CD19-N2 scFv:
(SEQ ID NO: 19)
GACATCCAGATGACACAGACTACATCCTCCCTGTCTGCCTCTCTGGGAGACAG
AGTCACCATCAGTTGCAGGGCAAGTCAGGACATTAGTAAATATTTAAATTGGTA
TCAGCAGAAACCAGATGGAACTGTTAAACTCCTGATCTACCATACATCAAGATT
ACACTCAGGAGTCCCATCAAGGTTCAGTGGCAGTGGGTCTGGAACAGATTATT
CTCTCACCATTAGCAACCTGGAGCAAGAAGATATTGCCACTTACTTTTGCCAAC
AGGGTAATACGCTTCCGTACACGTTCGGAGGGGGGACTAAGTTGGAAATAACA
CGGGCTGGTGGCGGTGGCTCGGGCGGTGGTGGGTCGGGTGGCGGCGGATCTG
AGGTGAAACTGCAGGAGTCAGGACCTGGCCTGGTGGCGCCCTCACAGAGCCT
GTCCGTCACATGCACTGTCTCAGGGGTCTCATTACCCGACTATGGTGTAAGCTG
GATTCGCCAGCCTCCACGAAAGGGTCTGGAGTGGCTGGGAGTAATATGGGGTA
GTGAAACCACATACTATAATTCAGCTCTCAAATCCAGACTGACCATCATCAAGG
ACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGG
ACTACTGGGGCCAAGGAACCTCAGTCACCGTCTCCTCA
Amino acid sequence of CD19-N2 scFv:
(SEQ ID NO: 20)
DIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITRAGGG
GSGGGGSGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIYYCAKH
YYYGGSYAMDYWGQGTSVTVSS
Nucleotide sequence of CD19-N3 scFv:
(SEQ ID NO: 21)
GACATCCAGATGACACAGACTACATCCTCCCTGTCTGCCTCTCTGGGAGACAG
AGTCACCATCAGTTGCAGGGCAAGTCAGGACATTAGTAAATATTTAAATTGGTA
TCAGCAGAAACCAGATGGAACTGTTAAACTCCTGATCTACCATACATCAAGATT
ACACTCAGGAGTCCCATCAAGGTTCAGTGGCAGTGGGTCTGGAACAGATTATT
CTCTCACCATTAGCAACCTGGAGCAAGAAGATATTGCCACTTACTTTTGCCAAC
AGGGTAATACGCTTCCGTACACGTTCGGAGGGGGGACTAAGTTGGAAATAACA
CGGGCGGCCGCAGGTGGCGGTGGCTCGGGCGGTGGTGGGTCGGGTGGCGGC
GGATCTGAGGTGAAACTGCAGGAGTCAGGACCTGGCCTGGTGGCGCCCTCAC
AGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAAT
ATGGGGTAGTGAAACCACATACTATAATTCAGCTCTCAAATCCAGACTGACCAT
CATCAAGGACAACTCCAAGAGCCAAGTTTTCTTAAAAATGAACAGTCTGCAA
ACTGATGACACAGCCATTTACTACTGTGCCAAACATTATTACTACGGTGGTAGC
TATGCTATGGACTACTGGGGCCAAGGAACCTCAGTCACCGTCTCCTCA
Amino acid sequence of CD19-N3 scFv:
(SEQ ID NO: 22)
DIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITRAAAG
GGGSGGGGSGGGGSEVKLQESGPGLVAPSQSLSVTCTVSGVSLPDYGVSWIRQPP
RKGLEWLGVIWGSETTYYNSALKSRLTIIKDNSKSQVFLKMNSLQTDDTAIYYCA
KHYYYGGSYAMDYWGQGTSVTVSS
Nucleotide sequence of CD19-N4 scFv:
(SEQ ID NO: 23)
GACATCCAGATGACACAGACTACATCCTCCCTGTCTGCCTCTCTGGGAGACAG
AGTCACCATCAGTTGCAGGGCAAGTCAGGACATTAGTAAATATTTAAATTGGTA
TCAGCAGAAACCAGATGGAACTGTTAAACTCCTGATCTACCATACATCAAGATT
ACACTCAGGAGTCCCATCAAGGTTCAGTGGCAGTGGGTCTGGAACAGATTATT
CTCTCACCATTAGCAACCTGGAGCAAGAAGATATTGCCACTTACTTTTGCCAAC
AGGGTAATACGCTTCCGTACACGTTCGGAGGGGGGACTAAGTTGGAAATAACA
CGGGCTGATGCTGCACCAACTGTATCCATCTTCCCACCAGGTGGCGGTGGCTC
GGGCGGTGGTGGGTCGGGTGGCGGCGGATCTGAGGTGAAACTGCAGGAGTCA
GGACCTGGCCTGGTGGCGCCCTCACAGAGCCTGTCCGTCACATGCACTGTCTC
AGGGGTCTCATTACCCGACTATGGTGTAAGCTGGATTCGCCAGCCTCCACGAA
AGGGTCTGGAGTGGCTGGGAGTAATATGGGGTAGTGAAACCACATACTATAAT
TCAGCTCTCAAATCCAGACTGACCATCATCAAGGACAACTCCAAGAGCCAAG
TTTTCTTAAAAATGAACAGTCTGCAAACTGATGACACAGCCATTTACTACTGTG
CCAAACATTATTACTACGGTGGTAGCTATGCTATGGACTACTGGGGCCAAGGAA
CCTCAGTCACCGTCTCCTCA
Amino acid sequence of CD19-N4 scFv:
(SEQ ID NO: 24)
DIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITRADAA
PTVSIFPPGGGGSGGGGSGGGGSEVKLQESGPGLVAPSQSLSVTCTVSGVSLPDYG
VSWIRQPPRKGLEWLGVIWGSETTYYNSALKSRLTIIKDNSKSQVFLKMNSLQTD
DTAIYYCAKHYYYGGSYAMDYWGQGTSVTVSS
Nucleotide sequence of CD19 CAR-N1:
(SEQ ID NO: 25)
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTC
TGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTA
CTTTTGCCAACAGGGTAATACGCTTCCGTACACGTTCGGAGGGGGGACTAAGT
TGGAAATAACAGGTGGCGGTGGCTCGGGCGGTGGTGGGTCGGGTGGCGGCGG
ATCTGAGGTGAAACTGCAGGAGTCAGGACCTGGCCTGGTGGCGCCCTCACAG
AGCCTGTCCGTCACATGCACTGTCTCAGGGGTCTCATTACCCGACTATGGTGTA
AGCTGGATTCGCCAGCCTCCACGAAAGGGTCTGGAGTGGCTGGGAGTAATATG
GGGTAGTGAAACCACATACTATAATTCAGCTCTCAAATCCAGACTGACCATCAT
CAAGGACAACTCCAAGAGCCAAGTTTTCTTAAAAATGAACAGTCTGCAAACT
GATGACACAGCCATTTACTACTGTGCCAAACATTATTACTACGGTGGTAGCTAT
GCTATGGACTACTGGGGCCAAGGAACCTCAGTCACCGTCTCCTCAACCACGAC
GCCAGCGCCGCGACCACCAACACCGGCGCCCACCATCGCGTCGCAGCCCCTG
TCCCTGCGCCCAGAGGCGTGCCGGCCAGCGGCGGGGGGCGCAGTGCACACG
AGGGGGCTGGACTTCGCCTGTGATATCTACATCTGGGCGCCCTTGGCCGGGAC
TTGTGGGGTCCTTCTCCTGTCACTGGTTATCACCCTTTACTGCAAACGGGGCA
GAAAGAAACTCCTGTATATATTCAAACAACCATTTATGAGACCAGTACAAACTA
CTCAAGAGGAAGATGGCTGTAGCTGCCGATTTCCAGAAGAAGAAGAAGGAGG
ATGTGAACTGAGAGTGAAGTTCAGCAGGAGCGCAGACGCCCCCGCGTACCAG
CAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCT
ACGACGCCCTTCACATGCAGGCCCTGCCCCCTCGCTGATAA
Amino acid sequence of CD19 CAR-N1:
(SEQ ID NO: 26)
MALPVTALLLPLALLLHAARPDIQMTQTTSSLSASLGDRVTISCRASQDISKYLN
WYQQKPDGTVKLLIYHTSRLHSGVPSRFSGSGSGTDYSLTISNLEQEDIATYFCQQ
GNTLPYTFGGGTKLEITGGGGSGGGGSGGGGSEVKLQESGPGLVAPSQSLSVTCT
VSGVSLPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRPPTPA
PTIASQPLSLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
Nucleotide sequence of CD19 CAR-N2:
(SEQ ID NO: 27)
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTC
TGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTA
CTTTTGCCAACAGGGTAATACGCTTCCGTACACGTTCGGAGGGGGGACTAAGT
TGGAAATAACACGGGCTGGTGGCGGTGGCTCGGGCGGTGGTGGGTCGGGTGG
CGGCGGATCTGAGGTGAAACTGCAGGAGTCAGGACCTGGCCTGGTGGCGCCC
TCACAGAGCCTGTCCGTCACATGCACTGTCTCAGGGGTCTCATTACCCGACTAT
GGTGTAAGCTGGATTCGCCAGCCTCCACGAAAGGGTCTGGAGTGGCTGGGAG
TAATATGGGGTAGTGAAACCACATACTATAATTCAGCTCTCAAATCCAGACTGA
CCATCATCAAGGACAACTCCAAGAGCCAAGTTTTCTTAAAAATGAACAGTCTG
CAAACTGATGACACAGCCATTTACTACTGTGCCAAACATTATTACTACGGTGGT
AGCTATGCTATGGACTACTGGGGCCAAGGAACCTCAGTCACCGTCTCCTCAAC
CACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGC
ACACGAGGGGGCTGGACTTCGCCTGTGATATCTACATCTGGGCGCCCTTGGCC
GGGACTTGTGGGGTCCTTCTCCTGTCACTGGTTATCACCCTTTACTGCAAACG
GGGCAGAAAGAAACTCCTGTATATATTCAAACAACCATTTATGAGACCAGTAC
AAACTACTCAAGAGGAAGATGGCTGTAGCTGCCGATTTCCAGAAGAAGAAGA
AGGAGGATGTGAACTGAGAGTGAAGTTCAGCAGGAGCGCAGACGCCCCCGC
GTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGG
GAAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAG
GACACCTACGACGCCCTTCACATGCAGGCCCTGCCCCCTCGCTGATAA
Amino acid sequence of CD19 CAR-N2:
(SEQ ID NO: 28)
MALPVTALLLPLALLLHAARPDIQMTQTTSSLSASLGDRVTISCRASQDISKYLN
WYQQKPDGTVKLLIYHTSRLHSGVPSRFSGSGSGTDYSLTISNLEQEDIATYFCQQ
GNTLPYTFGGGTKLEITRAGGGGSGGGGSGGGGSEVKLQESGPGLVAPSQSLSVT
CTVSGVSLPDYGVSWIRQPPRKGLEWLGVIWGSETTYYNSALKSRLTIIKDNSKS
QVFLKMNSLQTDDTAIYYCAKHYYYGGSYAMDYWGQGTSVTVSSTTTPAPRPPT
PAPTIASQPLSLRPEACRPAAGGAVHTRGLDFACDIYIWAPLAGTCGVLLLSLVITL
YCKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
Nucleotide sequence of CD19 CAR-N3:
(SEQ ID NO: 29)
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTC
TGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTA
CTTTTGCCAACAGGGTAATACGCTTCCGTACACGTTCGGAGGGGGGACTAAGT
TGGAAATAACACGGGCGGCCGCAGGTGGCGGTGGCTCGGGCGGTGGTGGGTC
GGGTGGCGGCGGATCTGAGGTGAAACTGCAGGAGTCAGGACCTGGCCTGGTG
GCGCCCTCACAGAGCCTGTCCGTCACATGCACTGTCTCAGGGGTCTCATTACC
CGACTATGGTGTAAGCTGGATTCGCCAGCCTCCACGAAAGGGTCTGGAGTGGC
TGGGAGTAATATGGGGTAGTGAAACCACATACTATAATTCAGCTCTCAAATCCA
GACTGACCATCATCAAGGACAACTCCAAGAGCCAAGTTTTCTTAAAAATGAAC
AGTCTGCAAACTGATGACACAGCCATTTACTACTGTGCCAAACATTATTACTAC
GGTGGTAGCTATGCTATGGACTACTGGGGCCAAGGAACCTCAGTCACCGTCTC
CTCAACCACGACGCCAGCGCCGCGACCACCAACACCGGCGCCCACCATCGCG
TCGCAGCCCCTGTCCCTGCGCCCAGAGGCGTGCCGGCCAGCGGCGGGGGGCG
CAGTGCACACGAGGGGGCTGGACTTCGCCTGTGATATCTACATCTGGGCGCCC
TTGGCCGGGACTTGTGGGGTCCTTCTCCTGTCACTGGTTATCACCCTTTACTGC
AAACGGGGCAGAAAGAAACTCCTGTATATATTCAAACAACCATTTATGAGACC
AGTACAAACTACTCAAGAGGAAGATGGCTGTAGCTGCCGATTTCCAGAAGAA
GAAGAAGGAGGATGTGAACTGAGAGTGAAGTTCAGCAGGAGCGCAGACGCC
CCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCAC
CAAGGACACCTACGACGCCCTTCACATGCAGGCCCTGCCCCCTCGCTGATAA
Amino acid sequence of CD19 CAR-N3:
(SEQ ID NO: 30)
MALPVTALLLPLALLLHAARPDIQMTQTTSSLSASLGDRVTISCRASQDISKYLN
WYQQKPDGTVKLLIYHTSRLHSGVPSRFSGSGSGTDYSLTISNLEQEDIATYFCQQ
GNTLPYTFGGGTKLEITRAAAGGGGSGGGGSGGGGSEVKLQESGPGLVAPSQSLS
VTCTVSGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSTTTPAPR
PPTPAPTIASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Nucleotide sequence of CD19 CAR-N4:
(SEQ ID NO: 31)
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTC
TGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTA
CTTTTGCCAACAGGGTAATACGCTTCCGTACACGTTCGGAGGGGGGACTAAGT
TGGAAATAACACGGGCTGATGCTGCACCAACTGTATCCATCTTCCCACCAGGT
GGCGGTGGCTCGGGCGGTGGTGGGTCGGGTGGCGGCGGATCTGAGGTGAAAC
TGCAGGAGTCAGGACCTGGCCTGGTGGCGCCCTCACAGAGCCTGTCCGTCAC
ATGCACTGTCTCAGGGGTCTCATTACCCGACTATGGTGTAAGCTGGATTCGCCA
GCCTCCACGAAAGGGTCTGGAGTGGCTGGGAGTAATATGGGGTAGTGAAACC
ACATACTATAATTCAGCTCTCAAATCCAGACTGACCATCATCAAGGACAACTCC
AAGAGCCAAGTTTTCTTAAAAATGAACAGTCTGCAAACTGATGACACAGCCAT
TTACTACTGTGCCAAACATTATTACTACGGTGGTAGCTATGCTATGGACTACTGG
GGCCAAGGAACCTCAGTCACCGTCTCCTCAACCACGACGCCAGCGCCGCGAC
CACCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCT
CCTGTCACTGGTTATCACCCTTTACTGCAAACGGGGCAGAAAGAAACTCCTGT
ATATATTCAAACAACCATTTATGAGACCAGTACAAACTACTCAAGAGGAAGAT
GGCTGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCAC
ATGCAGGCCCTGCCCCCTCGCTGATAA
Amino acid sequence of CD19 CAR-N4:
(SEQ ID NO: 32)
MALPVTALLLPLALLLHAARPDIQMTQTTSSLSASLGDRVTISCRASQDISKYLN
WYQQKPDGTVKLLIYHTSRLHSGVPSRFSGSGSGTDYSLTISNLEQEDIATYFCQQ
GNTLPYTFGGGTKLEITRADAAPTVSIFPPGGGGSGGGGSGGGGSEVKLQESGPG
LVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVS
STTTPAPRPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR

For the method of preparation of the positive control CTL109, see Patent Application WO2012079000A1, of which the application date is 9 Dec. 2011 and the publication date is 14 Jun. 2012. The sequences of the CD19-CAR in the Patent Application are as follows:

CD19-CAR nucleotide sequence of the positive control CTL109:
(SEQ ID NO: 33)
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTC
TGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTA
CTTTTGCCAACAGGGTAATACGCTTCCGTACACGTTCGGAGGGGGGACCAAGC
TGGAGATCACAGGTGGCGGTGGCTCGGGCGGTGGTGGGTCGGGTGGCGGCGG
ATCTGAGGTGAAACTGCAGGAGTCAGGACCTGGCCTGGTGGCGCCCTCACAG
AGCCTGTCCGTCACATGCACTGTCTCAGGGGTCTCATTACCCGACTATGGTGTA
AGCTGGATTCGCCAGCCTCCACGAAAGGGTCTGGAGTGGCTGGGAGTAATATG
GGGTAGTGAAACCACATACTATAATTCAGCTCTCAAATCCAGACTGACCATCAT
CAAGGACAACTCCAAGAGCCAAGTTTTCTTAAAAATGAACAGTCTGCAAACT
GATGACACAGCCATTTACTACTGTGCCAAACATTATTACTACGGTGGTAGCTAT
GCTATGGACTACTGGGGCCAAGGAACCTCAGTCACCGTCTCCTCAACCACGAC
GCCAGCGCCGCGACCACCAACACCGGCGCCCACCATCGCGTCGCAGCCCCTG
TCCCTGCGCCCAGAGGCGTGCCGGCCAGCGGCGGGGGGCGCAGTGCACACG
AGGGGGCTGGACTTCGCCTGTGATATCTACATCTGGGCGCCCTTGGCCGGGAC
TTGTGGGGTCCTTCTCCTGTCACTGGTTATCACCCTTTACTGCAAACGGGGCA
GAAAGAAACTCCTGTATATATTCAAACAACCATTTATGAGACCAGTACAAACTA
CTCAAGAGGAAGATGGCTGTAGCTGCCGATTTCCAGAAGAAGAAGAAGGAGG
ATGTGAACTGAGAGTGAAGTTCAGCAGGAGCGCAGACGCCCCCGCGTACAAG
CAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCT
ACGACGCCCTTCACATGCAGGCCCTGCCCCCTCGCTAA
Nucleotide sequence of CD3ζ intracellular region:
(SEQ ID NO: 56)
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTTTT
GGACAAGAGACGTGGCCGGGACCCTGAGATGGGGGGAAAGCCGAGAAGGAA
GAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCT
TCACATGCAGGCCCTGCCCCCTCGC
Amino acid sequence of CD3ζ intracellular region:
(SEQ ID NO: 57)
RVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHM
QALPPR

For the method of the preparation of the positive control CTL-019 of the present disclosure, see the Patent Application WO2012079000A1, in which the CD19-CAR nucleotide sequence of the positive control CTL-109 is shown in SEQ ID NO: 33, and the CD19-CAR amino acid sequence of the positive control CTL-019 is in SEQ ID NO: 34.

CD19-CAR amino acid sequence of the positive
control CTL109:
(SEQ ID NO: 34)
MALPVTALLLPLALLLHAARPDIQMTQTTSSLSASLGDRVTISCRASQD
ISKYLNWYQQKPDGTVKLLIYHTSRLHSGVPSRFSGSGSGTDYSLTISN
LEQEDIATYFCQQGNTLPYTFGGGTKLEITGGGGSGGGGSGGGGSEVKL
QESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAA
GGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQN
QLYNELNLGRREEYDVLDKRRGRDPEMGGKPRRKNPQEGLYNELQKDKM
AEAYSEIGMKGERRRGKGHDGLYQGLSTATKDTYDALHMQALPPR

For the method of preparation of the negative control CART-MSN, see Patent Application CN104159909A. During the process of preparing SS1CAR, the BamHI restriction site of SS1CAR (which was located between the CD8α leading signal region and SS1 scFv) taught in the Patent Application was removed when synthesizing the whole gene sequence of SS1-CAR. The nucleotide sequence of MSN-CAR (SS1CAR) is shown in SEQ ID NO: 35, and the amino acid sequence of MSN-CAR (SS1CAR) is shown in SEQ ID NO: 36.

Nucleotide sequence of MSN-CAR (SS1CAR):
(SEQ ID NO: 35)
ATGGCCTTACCAGTGACCGCCTTGCTCCTGCCGCTGGCCTTGCTGCTCC
ACGCCGCCAGGCCGCAGGTACAACTGCAGCAGTCTGGGCCTGAGCTGGA
GAAGCCTGGCGCTTCAGTGAAGATATCCTGCAAGGCTTCTGGTTACTCA
TTCACTGGCTACACCATGAACTGGGTGAAGCAGAGCCATGGAAAGAGCC
TTGAGTGGATTGGACTTATTACTCCTTACAATGGTGCTTCTAGCTACAA
CCAGAAGTTCAGGGGCAAGGCCACATTAACTGTAGACAAGTCATCCAGC
ACAGCCTACATGGACCTCCTCAGTCTGACATCTGAAGACTCTGCAGTCT
ATTTCTGTGCAAGGGGGGGTTACGACGGGAGGGGTTTTGACTACTGGGG
CCAAGGGACCACGGTCACCGTCTCCTCAGGTGGAGGCGGTTCAGGCGGC
GGTGGCTCTAGCGGTGGTGGATCGGACATCGAGCTCACTCAGTCTCCAG
CAATCATGTCTGCATCTCCAGGGGAGAAGGTCACCATGACCTGCAGTGC
CAGCTCAAGTGTAAGTTACATGCACTGGTACCAGCAGAAGTCAGGCACC
TCCCCCAAAAGATGGATTTATGACACATCCAAACTGGCTTCTGGAGTCC
CAGGTCGCTTCAGTGGCAGTGGGTCTGGAAACTCTTACTCTCTCACAAT
CAGCAGCGTGGAGGCTGAAGATGATGCAACTTATTACTGCCAGCAGTGG
AGTAAGCACCCTCTCACGTACGGTGCTGGGACAAAGTTGGAAATCAAAA
GCAGCGAGAGCAAGTACGGCCCTCCCTGCCCCCCTTGCCCTGCCCCCGA
GTTCCTGGGCGGACCCAGCGTGTTCCTGTTCCCCCCCAAGCCCAAGGAC
ACCCTGATGATCAGCCGGACCCCCGAGGTGACCTGTGTGGTGGTGGACG
TGTCCCAGGAGGACCCCGAGGTCCAGTTCAACTGGTACGTGGACGGCGT
GGAGGTGCACAACGCCAAGACCAAGCCCCGGGAGGAGCAGTTCAATAGC
ACCTACCGGGTGGTGTCCGTGCTGACCGTGCTGCACCAGGACTGGCTGA
ACGGCAAGGAATACAAGTGTAAGGTGTCCAACAAGGGCCTGCCCAGCAG
CATCGAGAAAACCATCAGCAAGGCCAAGGGCCAGCCTCGGGAGCCCCAG
GTGTACACCCTGCCCCCTAGCCAAGAGGAGATGACCAAGAACCAGGTGT
CCCTGACCTGCCTGGTGAAGGGCTTCTACCCCAGCGACATCGCCGTGGA
GTGGGAGAGCAACGGCCAGCCCGAGAACAACTACAAGACCACCCCCCCT
GTGCTGGACAGCGACGGCAGCTTCTTCCTGTACAGCCGGCTGACCGTGG
ACAAGAGCCGGTGGCAGGAGGGCAACGTCTTTAGCTGCTCCGTGATGCA
CGAGGCCCTGCACAACCACTACACCCAGAAGAGCCTGAGCCTGTCCCTG
GGCAAGATGTTCTGGGTGCTGGTCGTTGTGGGCGGCGTGCTGGCCTGCT
ACAGCCTGCTGGTGACAGTGGCCTTCATCATCTTTTGGGTGAGGAGCAA
GCGGAGCAGACTGCTGCACAGCGACTACATGAACATGACCCCCCGGAGG
CCTGGCCCCACCCGGAAGCACTACCAGCCCTACGCCCCTCCCAGGGATT
TCGCCGCCTACCGGAGCCGGGTGAAGTTCAGCCGGAGCGCCGACGCCCC
TGCCTACCAGCAGGGCCAGAACCAGCTGTACAACGAGCTGAACCTGGGC
CGGAGGGAGGAGTACGACGTGCTGGACAAGCGGAGAGGCCGGGACCCTG
AGATGGGCGGCAAGCCCCGGAGAAAGAACCCCCAGGAGGGCCTGTATAA
CGAACTGCAGAAAGACAAGATGGCCGAGGCCTACAGCGAGATCGGCATG
AAGGGCGAGCGGAGGCGGGGCAAGGGCCACGACGGCCTGTACCAGGGCC
TGAGCACCGCCACCAAGGATACCTACGACGCCCTGCACATGCAGGCCCT
GCCCCCCAGA
Amino acid sequence of MSN-CAR (SS1CAR):
(SEQ ID NO: 36)
MALPVTALLLPLALLLHAARPQVQLQQSGPELEKPGASVKISCKASGYS
FTGYTMNWVKQSHGKSLEWIGLITPYNGASSYNQKFRGKATLTVDKSSS
TAYMDLLSLTSEDSAVYFCARGGYDGRGFDYWGQGTTVTVSSGGGGSGG
GGSSGGGSDIELTQSPAIMSASPGEKVTMTCSASSSVSYMHWYQQKSGT
SPKRWIYDTSKLASGVPGRFSGSGSGNSYSLTISSVEAEDDATYYCQQW
SKHPLTYGAGTKLEIKSSESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSL
GKMFWVLVVVGGVLACYSLLVTVAFIIFWVRSKRSRLLHSDYMNMTPRR
PGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGM
KGERRRGKGHDGLYQGLSTATKDTYDALHMQALPPR

### Example 2. Extraction of PBMCs

Healthy volunteers who had no symptom of cold or fever were recruited, and informed consents were signed. 100 ml of blood was taken from a vein by medical professionals to BD anticoagulant tubes (Cat. No. 367886). Blood was mixed with an equal amount of PBS buffer (containing 2% fetal bovine serum). 15 ml of Ficoll Buffer (GE healthcare, 17-5442-02) was added into PBMC-Separation Tube Sepmate-50 (STEMCELL Technology, Cat. No. 86450), and added with the mixture of blood and PBS. The pellet was resuspended in PBS after centrifugation. The resuspended cells were counted, and 10 μl of the suspension was taken out, added with 10 μl of 0.1% Trypan blue and mixed well. The cells were counted and survival rate was calculated.

### Example 3. Purification of T cells

PBMC cells were centrifuged at 300 g for 5 minutes, the supernatant was discarded, and the cells were resuspended by adding the corresponding amount of PBS buffer (containing 2 mM EDTA and 1% fetal calf serum), and the cell density was adjusted to 5×10⁷/ml. Human T cells were purified by using EasySep™ Human T Cell Enrichment Kit (STEMCELL, Cat. No. 17951). First, 50 μl/ml of Cooktail Protease Inhibitor (Biotool, B14001a) was added to PBMC suspension, mixed well and allowed to stand at room temperature for 10 minutes. Then, 50 μl/ml of EasySep™ D Magnetite Particles (STEMCELL, Cat. No. 19550) was added and mixed well, and allowed to stand at room temperature for 5 minutes. The cell suspension was added into a 5 ml flow tube and placed in magnetic pole for 5 minutes. The cell suspension was quickly decanted, PBS buffer was supplemented into the flow tube and resuspended, and the steps were repeated for 3 times. The resulting cell suspension was centrifuged at 300 g for 5 minutes, the supernatant was discarded, the cell pellet was resuspended in VIVO-15 medium (LONZA), and the density was adjusted to 1×10⁶/ml. Then rIL-2 (R&D, Cat. No.: 202-IL-050) was added at a concentration of 100 IU/ml, and cultured in cell culture incubator at 37° C.

### Example 4. Activation of T cells

Anti-CD3/anti-CD28 magnetic beads (Life Technology, Cat. No. 11131D) were resuspended in PBS buffer (containing 2 mM EDTA and 1% fetal bovine serum), then placed in a magnetic pole for 2 minutes, and the supernatant was discarded. The above steps were repeated for 4 times. The magnetic beads were washed, added with the purified T cells at a ratio of 1:1 (by magnetic bead number), mixed well, and cultured at 37° C. for 3 days. After 3 days, the magnetic beads were taken out, and the target cells were resuspended several times with a pipette. The cell suspension was placed in a magnetic pole, allowed to stand for two minutes and the magnetic beads on the wall of the tube were discarded.

### Example 5. Infection of T Cells with pLVX-EF1-CD19 CAR Virus

(1) Packaging and concentration of pLVX-EF1-CD19 CAR Lentivirus:

Lentiviral plasmid pLVX-EF1-CD19 CAR and two helper plasmids, pCMV-dR8.91 (purchased from addgene) and pCMV-VSV-G (purchased from addgene), were extracted by using Maxi Plasmid Extraction Kit, Tiangen. One day before transfection, 293T cells (purchased from ATCC) were grown to full confluence in 75 cm² culture dishes, and passaged at 1:3 with 15 ml of culture medium per dish. Transfection was carried out according to Lipo3000 (Life technologies, Cat. No. L3000008). An exemplary transfection system is as follows:

| Transfection system 1 | Transfection system 2 |
|---|---|
| CART19-N2: 7.5 μg | CART19-N2: 7.5 μg |
| pCMV-dR8.91: 5.625 μg | pCMV-dR8.91: 5.625 μg |
| pCMV-VSV-G: 1.875 μg | pCMV-VSV-G: 1.875 μg |
| Opti-MEM (Gibco): 700 μl | Opti-MEM (Gibco): 700 μl |
| P3000: 30 μl | Lipofectamine: 36 μl |

The System 1 was well mixed with System 2, allowed to stand for 5 minutes, then the two systems were mixed again and allowed to stand for 10 minutes. 293T cells were carefully added. Fresh medium was replaced for the medium after 6 hours. After 48 hours, the culture medium was collected and stored at 4° C., and 15 ml of new Opti-MEM medium (Gibco, Cat. No. 51985034) was added again. The supernatant was collected again after 24 hours. The resulting virus supernatant was filtered through 0.45 μm filter, centrifuged in an ultracentrifuge tub at 50000 g for 2 hours and 45 minutes at 4° C. The supernatant was carefully removed thoroughly, and the white visible virus pellet was resuspended in PBS buffer at a volume of 1% of the supernatant. The resuspended virus was placed at 4° C. for about 30 minutes for dissolution. After complete dissolution, the virus solution was aliquoted and stored in a freezer at −80° C.

(2) Infection of T Cells with pLVX-EF1-CD19 CAR Lentivirus

The human primary T cells were activated with anti-CD3/anti-CD28 magnetic beads for 1 day, resuspended, placed in a magnetic pole for two minutes, and the cell suspension was taken out for cell counting. Approximately 1×10⁷ cells were centrifuged at 300 g for 5 minutes, the medium was discarded, added with 1 ml of fresh medium and resuspended. The concentrated lentivirus was added to adjust MOI to 5, mixed, centrifuged at 2000 g for 90 minutes at 32° C., the supernatant was discarded, and new medium Lonza X-VIVO 15 (containing 100 IU/ml rIL-2, purchased from R&D, Cat. No.: 202-IL-050) was added to adjust the cell density to 1×10⁶/ml, and added with the newly isolated anti-CD3/anti-CD28 magnetic beads after resuspension. The culture was continued in an incubator at 37° C. to obtain CD19 CAR-T cells (CART19): CART19-N2. The magnetic beads were removed by using magnetic separator before in vitro and in vivo tests.

CD19 CAR-T cells: CART19-N1, CART19-N3, and CART19-N4 were obtained in a similar way.

### Example 6. Knockout of TCR, B2M and PD-1 Genes in CART19 Cells

(1) crRNA Design

Appropriate target regions were selected on the basis of the nucleotide sequences of TRAC, B2M and PD-1, and crRNAs with 17-20 nt in length were designed. The resulting crRNAs were ligated with tracrRNA sequence corresponding to the Cas9 protein to form sgRNAs, wherein the crRNAs were located at 5′-end of the tracrRNA. CrRNAs with high knockout efficiency and low off-target rate were selected by experiments. Some crRNA sequences selected are as follows:

TABLE 1
crRNAs against the target gene

| Target | crRNA | | SEQ |
|---|---|---|---|
| gene | name | sequence | ID NO |
| TRAC | crRNA-1 | CAAAUGUGUCACAAAGUA | 37 |
| | crRNA-2 | AAAACUGUGCUAGACAUG | 38 |
| | crRNA-3 | UCAAGAGCAACAGUGCUG | 39 |
| | crRNA-4 | CACCUUCUUCCCCAGCCC | 40 |
| | crRNA-5 | GAAUAAUGCUGUUGUUGA | 41 |
| | crRNA-6 | GAUUUAGAGUCUCUCAGC | 42 |
| | crRNA-7 | ACGGCAGGGUCAGGGUUC | 43 |
| | crRNA-8 | GUUCCUGUGAUGUCAAGC | 44 |
| | crRNA-9 | UCAAAACCUGUCAGUGAU | 45 |
| | crRNA-10 | GAAUCCUCCUCCUGAAAG | 46 |
| | crRNA-11 | GGUACACGGCAGGGUCA | 47 |
| | crRNA-12 | GAGAAUCAAAAUCGGUGAAU | 48 |
| B2M | crRNA-13 | GUAGCGCGAGCACAGCUA | 49 |
| PD-1 | crRNA-14 | CGACUGGCCAGGGCGCCUGU | 50 |
| | crRNA-15 | GUGCUACAACUGGGCUGG | 51 |
| | crRNA-16 | GGCGCCCUGGCCAGUCGUCU | 52 |

The Cas9 protein is derived from Streptococcus Pyogenes (Cas9 Nuclease NLS, S. pyogenes (BioLabs)), the corresponding tracrRNA sequence is shown in SEQ ID NO: 53, and the amino acid sequence of Cas9 protein (including NLS) is shown in SEQ ID NO: 54.

SEQ ID NO: 53
GUUUUAGAGCUAGAAAUAGCAAGUUAAAAUAAGGCUAGUCCGUUAUCAA
CUUGAAAAAGUGGCACCGAGUCGGUGCUUUU
Amino acid sequence of Cas9 protein (including
NLS):
SEQ ID NO: 54
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGDPKKK
RKVMDK

(2) In Vitro Transcription of sgRNA:

First, PCR amplification was performed with sgRNA as a template. The PCR amplification system was shown in the following table.

TABLE 2
PCR amplification system with sgRNA as a template

| | |
|---|---|
| Reaction system (20 μl) | PCR condition |

| plasmid | 1 μl (0.5 pg) | | 98° C. | 30 | sec |
|---|---|---|---|---|---|
| 5 × HF buffer | 4 | μl | 98° C. | 10 | sec |
| dNTP (10 mM) | 0.4 | μl | 60° C. | 25 | sec |
| primer-F(10 μm) | 0.4 | μl | 72° C. | 2 | min |
| primer-R(10 μm) | 0.4 | μl | 72° C. | 10 | min |

| | | | |
|---|---|---|---|
| Phusion | 0.2 | μl | 38 times |

| | | | | | |
|---|---|---|---|---|---|
| H₂O | 13.6 | μl | 4° C. | 10 | min |

PCR Product Recovery:

See manual available from the Common DNA Product Purification Kit DP214, Tiangen. DNAs were obtained for in vitro transcription of sgRNAs. The transcription of sgRNAs was perfromed with Ambion in vitro Transcription Kit MEGAshortscript™ Kit (cat #AM1354). See manual available from Ambion MEGAclear™ Kit (cat #AM1908).

The resulting sgRNAs were purified and detected by spectrophotometer and denaturing agarose gel electrophoresis. All sgRNAs were qualified and aliquoted for use.

(3) Knockout of TRAC, B2M and PD-1 Gene was Performed in CART19 Cells by Transfection of CRISPR-Cas9 Via Electroporation

The resulting CART19 cells were subjected to electroporation by using LONZA 4D Electroporator (this method can also be used to knock out primary T cells), with the kit: P3 Primary Cell 4D-Nucleofector™ X kit (LONZA, V4XP3024).

First, the following electroporation system was prepared:

TABLE 3
Electroporation system

| | TCR, B2M, PD-1 | TCR/B2M | TCR/B2M/PD- |
|---|---|---|---|
| | single knockout | Double knockout | 1triple knockout |
| component | system | system | system |
| Nucleofector | 10 μl | 10 μl | 10 μl |
| buffer | | | |
| Cas9 protein | 30 μg | 30 μg | 30 μg |
| sgRNA | 4 μg | 4 μgTRAC + 4 | 4 μgTRAC + 4 |
| | | μg B2M | μg B2M + PD-1 |

The above electroporation system was mixed and incubated at room temperature for 10 minutes. The CAR-T cells were activated for three days, and then the anti-CD3/anti-CD28 magnetic beads were removed with magnetic poles. 5×10⁶ cells/tube were centrifuged at 300 g for 5 minutes, the supernatant was completely removed, the cell pellets were added with the pre-incubated electroporation system, together with 72 μl of Nucleofector buffer and 18 μl of Supplyment buffer, mixed, and added into a 100 μl LONZA electroporation cuvette. The cuvette was placed in LONZA-4D Electroporator and electroporation was performed according to E0-115 procedure. After the electroporation was finished, the electroporation cuvette was allowed to stand at room temperature for 5 minutes. The cells were transferred from the electroporation cuvette to pre-warmed X-VIVO-15 medium, cell density was adjusted to 1×10⁶/ml, and cultured at 37° C.

### Example 7. Screening of TCR and B2M Negative CART19 Cells

CART19 cells were cultured for 10 days after the knockout of TRAC by CRISPR-Cas9, and TCR-negative cells were enriched. All cells were first centrifuged at 300 g for 5 minutes and washed twice with PBS buffer (containing 2 mM EDTA and 1% fetal bovine serum). The cell density was adjusted to 1×10⁷ cells/ml, then 100 μl/ml of Biotin-TCR antibody (purchased from Miltenyi Germany, Cat. No. 130-109-918) was added, and incubated at 4° C. in the dark for 10 minutes. After Centrifugation at 300 g for 5 minutes, the cells were washed once with PBS buffer, and the cell density was adjusted to 1×10⁷ cells/ml. 50 μl/ml anti-Biotin Microbeads (purchased from Miltenyi, Cat. No. 130-090-485) were added and placed at 4° C. in the dark for 15 minutes. Centrifugation was performed at 300 g for 5 minutes, the cells were washed once with PBS buffer, and resuspended in 500 μl buffer. The LD column (purchased from Miltenyi, Cat. No. 130-042-901) was placed in magnetic poles, rinsed once with 2 ml PBS and loaded with 500 μl cell suspension. The target cells were collected after flowing through the LD column, and the column was then washed twice with 2 ml of PBS buffer. The collected target cell suspension was centrifuged at 300 g for 5 minutes and resuspended in pre-warmed medium, resulting in CART19 cells with knockout of TCR, i.e., UCART19^{TCR-/-}.

The enriched TCR-negative cells were washed twice with PBS buffer (containing 2 mM EDTA and 1% fetal bovine serum), the cell density was adjusted to 1×10⁷ cells/ml, then 100 l/ml of Biotin-B2M antibody (purchased from Miltenyi, Cat. No. 130-090-485) was added, and incubated at 4° C. in the dark for 10 minutes. After centrifugation at 300 g for 5 minutes, the cells were washed once again with PBS buffer, the cell density was re-adjusted to 1×10⁷ cells/ml, 50 μl/ml anti-Biotin Microbeads (purchased from Miltenyi, Cat. No. 130-090-485) was added and placed at 4° C. in the dark for 15 minutes. Centrifugation was performed at 300 g for 5 minutes, the cells were washed once with PBS buffer, and resuspended in 500 μl buffer. The LD column (purchased from Miltenyi, Cat. No. 130-042-901) was placed in magnetic poles, rinsed once with 2 ml PBS and loaded with 500 μl cell suspension. The target cells were collected after flowing through the LD column, and the column was then washed twice with 2 ml of PBS buffer. The collected target cell suspension was centrifuged at 300 g for 5 minutes and resuspended in pre-warmed medium for culture, resulting in CD19-CART cells with double knockout of both TCR and B2M, i.e., UCART19^{TCR-/-B2M-/-}. The CD19-CART cells with triple knockout of TCR, B2M and PD-1 (i.e., UCART^{TCR-/-B2M-/-PD-1-/-}) were screened in a similar way, and the identification of PD-1 knockout cells was shown in Test Example 5.

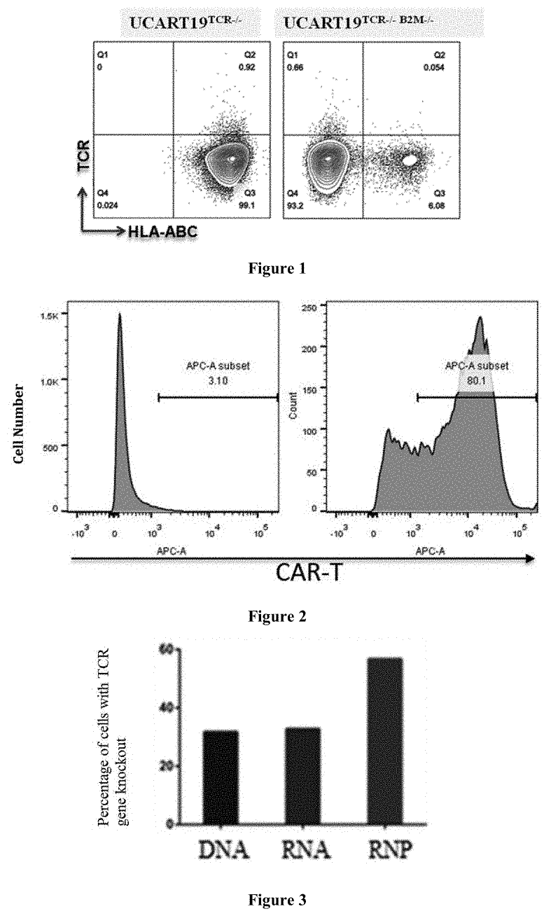

The enriched cells were sorted using BD Sorting Flow Cytometer, and the results of the purity of UCART^{TCR-/-} and UCART^{TCR-/-B2M-/-} were shown in FIG. 1. From the results, it can be seen that UCART^{TCR-/-} cells obtained by the above screening method have a purity up to 99% or more, and the UCART^{TCR-/-B2M-/-} cells have a purity up to 90% or more.

### Test Examples

### Test Example 1. Determination of Efficiency of pLVX-EF1-CD19 CAR Lentivirus in Transfecting T Cells

The T cells were transfected with the lentiviral plasmid pLVX-EF1-002B with MOI=5, mixed well, and centrifuged at 1600 g, 32° C. for 1.5 hours. After the centrifugation, the virus supernatant was carefully aspirated, and the density was adjusted to 5×10⁵ cells/ml with pre-warmed X-VIVO medium (containing 100 U/ml rhIL-2), and cultured in an incubator with carbon dioxide at 37° C. Four days later, the transfection efficiency was measured by flow cytometry by using Biotin-Protein L (Genscript Biotech., Cat. No. M00097), and the results were shown in FIG. 2.

From the results, it can be seen that the transfection efficiency was up to 80%, indicating that the above lentivirus transfection method can be used for the preparation of CART cells.

### Test Example 2. Analysis of TCR Knockout Efficiency with Various crRNA CRISPR-Cas9

The knockout efficiency was compared in the experiments by using the crRNAs against TRAC as designed in Example 6. After in vitro transcription, the resulting sgRNA and the Cas9 protein was electroporated into the activated primary T cells, and the expression of extracellular TCR protein was detected by flow cytometry 48 hours later. All the designed crRNAs can knock down the TRAC gene to varying degrees, and crRNA-11 has the highest knockout efficiency (data not shown).

### Test Example 3. Comparative Analysis of Different Delivery Systems

Three delivery systems: plasmid, mRNA and RNP (protein RNA complex).

CrRNA-11 is against TRAC, and large quantities of plasmids were extracted with Maxi Plasmid Extraction Kit, Tiangen.

In vitro transcription of Cas9 mRNA: First, a DNA template containing T7 promoter was obtained by PCR with T7 primer, and then Cas9 mRNA was obtained by in vitro transcription with T7 in vitro Transcription Kit, Ambion (Thermo, AM1345).

sgRNA and Cas9 protein complex were prepared according to Example 6.

5×10⁶ Jurkat cells (purchased from ATCC) were centrifuged to remove the supernatant, and then were transfected with three different delivery systems via Electroporation System Neon MPK5000, Invitrogen. After 48 hours, 0.5×10⁶ cells were washed twice with PBS buffer, and were resuspended in 100 μl of buffer, added with 10 μl of PE-TCR antibody (eBioscience, Cat. No. H57-597), mixed well, and incubated at 4° C. for 30 minutes. After washing once with PBS buffer, the cells were resuspended by adding 500 μl of buffer, and the expression level of TCR was detected by Flow Cytometry. The results were shown in FIG. 3.

The results indicate that the delivery system with sgRNA and Cas9 protein complex (RNP) can achieve the highest knockout efficiency and the system is a preferred method.

### Test Example 4. Random N-Oligo or Fish Sperm DNA Increases the TRAC Knockout Efficiency by CRISPR-Cas9

When gene knockout was performed using the RNP delivery system, RNP was mixed with random sequence of N-oligo (oligodeoxyribonucleic acid) or fish sperm DNA (R&D, Cat. No. 9610-5-D) and then was transfected via electroporation.

An exemplary N-oligo sequence:
(SEQ ID NO: 55)
TCATGTGGTCGGGGTAGCGGCTGAAGCACTGCACGCCGTACGTCAGGGT
GGTCACGAGGGTGGGCCAGGGCACGGGCAGCTTGCCGGTGGTGCAGATG
AACTTCAGGGTCAGCTTGCCGTAGGTGGC

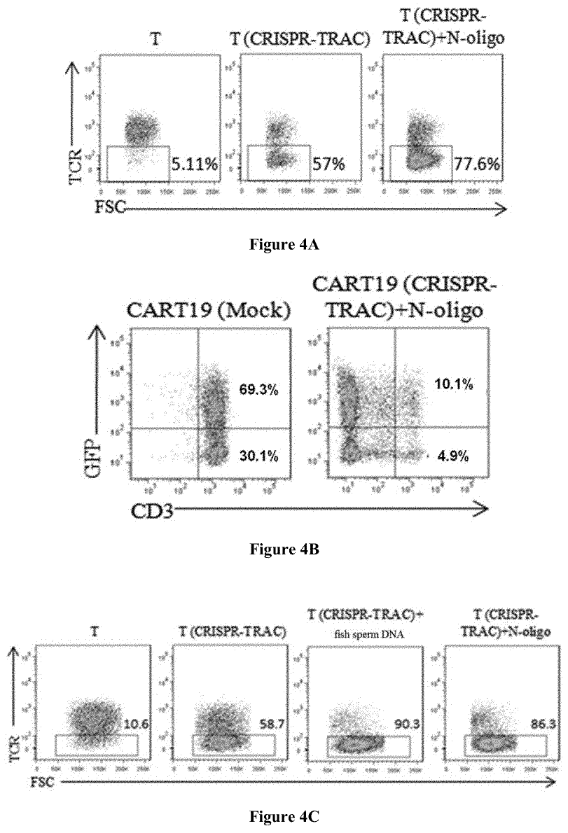

On the basis of Example 5 (3), 100-200 nM of N-oligo DNA was further added into the RNP complex, and the N-oligo DNA was Page Grade. The effect of N-oligo on TRAC knockout efficiency by CRISPR-Cas9 was shown in FIG. 4A and FIG. 4B. The results show that N-oligo can effectively increase TRAC gene knockout efficiency by CRISPR-Cas9 in both T cells and CART19 cells.

On the basis of Example 5 (3), 100-200 nM fish sperm DNA fragment was further added into the RNP complex, and the effect of the fish sperm DNA fragment on TRAC knockout efficiency was shown in FIG. 4C. The results showed that the TARC knockout efficiency was 90.3% by adding fish sperm DNA, while the TRAC gene knockout efficiency was 86.3% by adding N-oligo, indicating that the addition of fish sperm DNA fragment can improve the knockout efficiency of TRAC gene.

### Test Example 5. Detection of B2M and PD-1 Knockout Efficiency in T Cells

A number of crRNAs were designed in a similar way, and after a comparative analysis, the one with the highest knockout efficiency and the lowest off-target rate was selected for B2M gene knockout. The B2M and/or PD-1 gene of T cells were knocked out using RNP delivery system and N-oligo according to the same method as that in Example 5 (3).

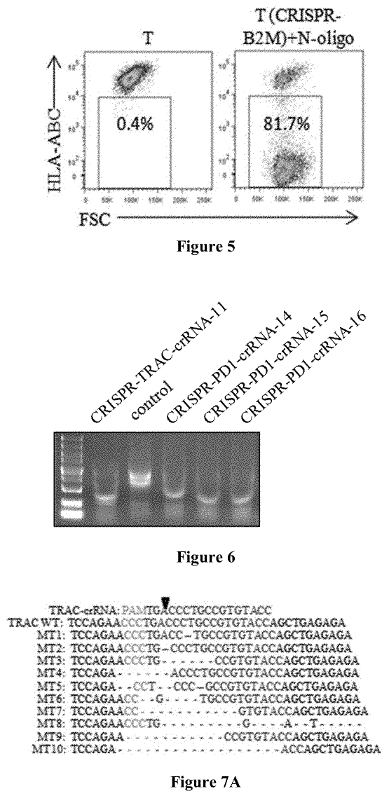

For B2M protein, the B2M gene expression was closely correlated with the display of HLA-ABC on the cell membrane, and the B2M gene knockdown efficiency was detected by using APC-HLA-ABC antibody (eBioscience, Cat. No. 12-9983-71). The results (FIG. 5) showed that the B2M gene knockout efficiency was greater than 80%.

For PD-1 gene, RNP and N-oligo were mixed and transfected into the cells via electroporation, 48 hours later, 1×10⁶ cells were washed twice with PBS buffer and the supernatant was completely aspirated. T7E1 experiment was performed according to the manual provided in GeneArt® Genomic Cleavage Detection Kit (Thermo Fisher). The knockout efficiency was calculated by comparing the optical density of PCR fragment corresponding to the intact wild-type gene to the optical density of two small fragments generated after mutation. The specific calculation formula was as follows:

Knockout efficiency=1−[(1−cleavage percentage)^{1/2}], wherein the cleavage percentage=the sum of optical density of the small fragments after cleavage/the sum of optical density of the small fragments after cleavage+the optical density of the un-cleaved fragment).

The results (shown in FIG. 6) showed that the selected three crRNAs against PD-1 can effectively knock out the PD-1 gene, all of them had the knockout efficiency of more than 80%, and crRNA-16 had the highest knockout efficiency.

### Test Example 6. Analysis of Gene Mutations Caused by CRISPR-Cas9

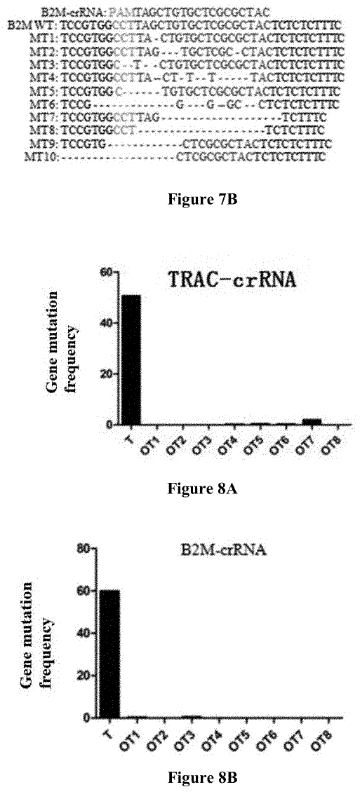

First, primers were designed near the target sites of the TRAC, B2M and PD-1 genes. For T cells, TRAC, B2M and PD-1 were knocked out by using RNP+N-oligo or fish sperm DNA fragments based on the CRISPR-Cas9 system. Genomic DNAs were extracted from 1×10⁶ normal T cells and gene knockout T cells, respectively. The resulting PCR product DNA fragment was ligated with the T blunt end vector (pEASY-Blunt Simple Cloning Kit, Beijing TransGen Biotech Co., Ltd., Cat. No. CB111-01). TOP10 competent cells were transformed with the ligation product and plated onto Amp-resistant solid plates. On the next day, the resulting clones were sequenced, and at least 30 clones per plate were tested. The obtained sequencing results were compared with wild-type sequences. The results were shown in FIGS. 7A-7B, in which the analysis results of PD-1 mutation were not shown and MT was the clone number.

From the results, it can be seen that for TRAC, B2M and PD-1, gene mutations were observed in the genomic DNAs corresponding to each crRNA, respectively, indicating that the TRAC, B2M and PD-1 genes were actually knocked out at the gene level.

### Test Example 7. Off-Target Analysis

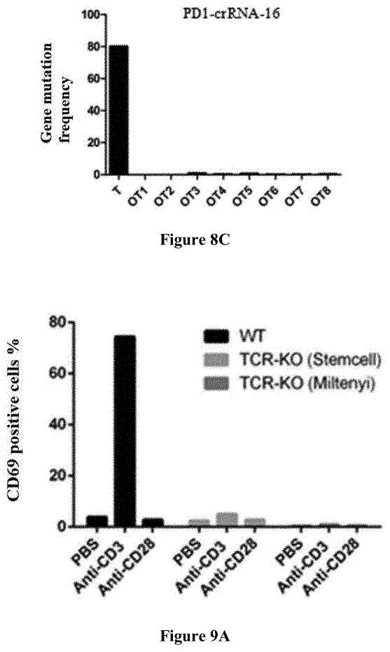

The off-target sites which might occur were predicted for the designed crRNAs (crRNA-11, crRNA-13 and crRNA-16) on http://crispr.mit.edu/. For each of TRAC, B2M and PD-1, 8-9 potential off-target sites (OT1-OT9) were selected and primers against these potential off-target sites were designed for PCR amplification. The peak maβ sequencing results of the off-target sites of the genomic DNA in gene knockout cells and the that of control (target gene TRAC, B2M or PD-1) were subjected to TIDE alignment analysis on https://tide.nki.nl/ with. The results (shown in FIGS. 8A-8C) showed the off-target rates were extremely low with the knockout method and the selected crRNAs against TRAC, B2M and PD-1.

### Test Example 8. Analysis of the Effect of TCR Knockout on Cell Signaling Pathway and Killing Activity

96-well plates were coated with CD3 antibody (5 μg/ml) or CD28 antibody (5 g/ml), 100 μl was added into per well, and coated at 37° C. for two hours. The plates were then washed twice with PBS, added with TCR-negative T cells and normal T cells respectively, at a cell density of 1×10⁶ cells/ml. After incubation at 37° C. for 24 hours, cells were stained with CD25 and CD69-antibodies, and the expression of CD25 and CD69 was detected by Flow Cytometry.

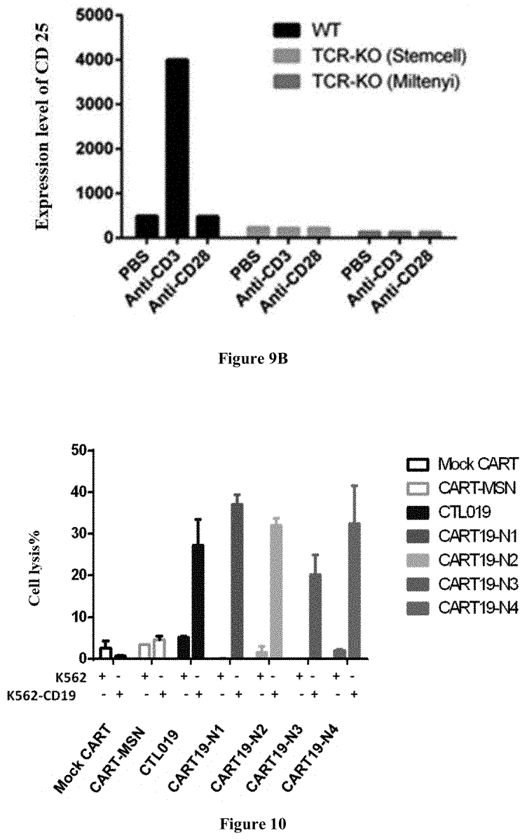

The results (shown in FIGS. 9A-9B) indicated that T cells, after TRAC gene knockout, cannot be induced to express CD25 and CD69 by CD3 or CD28 antibody, i.e., CATR cells with knockout of TCR cannot be activated by CD3 antibody or CD28.

### Test Example 9. Analysis of In Vitro Cytotoxicity of CD19-CART Cells

Materials: K562, Raji and Daudi cells were purchased from ATCC, Nalm6 was purchased from BD, Human IL-2 ELISA Kit II (Cat. No. 550611) and Human IFN-7 ELISA Kit II (Cat. No. 550612) were purchased from BD, and anti-human CD107a (Cat. No. 555801) antibody was purchased from BD.

Method and Result:

9.1 In Vitro Killing of K562-CD19 Cells by CART Cells

The K562-CD19 cells were constructed as follows: CD19 antigen was designed with reference to the NCBI NM_001770.5 sequence, and was constructed into pLVX-EF1-CD19 plasmid. K562 cells were transfected and a single clone was picked up to obtain a K562-CD19 cell line.

The target cells (K562-CD19 cells and K562 cells) were adjusted to a density of 5×10⁵/ml, 100 μl of which were plated into a 96-well round bottom plate, added with effector cells at a range of ratio of effector cell (CART19) to target cell (E:T ratio=30:1 to 0.3:1) or at a specific ratio (30:1), and mixed by pipetting. Centrifugation was performed at 1000 rpm for 2 min, cell lysis was detected after incubation in an incubator for 4 h. 150 μl of the supernatant was collected and frozen at −20° C. for subsequent experiments. 50 μl of supernatant was maintained in each well, added with 1001 of detection solution (Steady-Glo® Luciferase Assay System, E2520, Promega), incubated at room temperature for 5 min, and 100 μl was pipetted into a black plate. The bioluminescence value was measured by using a microplate reader, and the killing rate was calculated.

Killing rate=(Reading of Target only−Reading of Target plus Effector)/Reading of Target only.

The killing results were shown in FIG. 10, in which Mock CART indicated T cells containing a vehicle, and CART-MSN indicated CART cells against mesothelin. It can be seen from the results that CART19 cells had significant killing effect on K562-CD19 cells, but had no killing effect on K562 cells (CD19 negative cells), indicating that CART19 cells can specifically kill CD19-positive cells.

9.2 In Vitro Killing of Raji Cells by CART Cells

The target cells (Raji cells) were adjusted to a density of 5×10⁵/ml, 100 μl of which were plated into a 96-well round bottom plate, added with effector cells (CART19) at a range of ratio of effector cell to target cell (E:T ratio=30:1 to 0.3:1; 30:1, 10:1, 3:1, 1:1 and 0.3:1), and mixed by pipetting. CTL-019 cells were used as positive control and centrifuged at 1000 rpm for 2 min, incubated in an incubator for 4 h, and then cell lysis was detected. 150 μl of the supernatant was collected and frozen at −20° C. for subsequent experiments. 50 μl of supernatant was maintained in each well, added with 100 μl of detection solution (Steady-Glo® Luciferase Assay System, E2520, Promega), incubated at room temperature for 5 min, and 100 μl was pipetted into a black plate. The bioluminescence value was measured by using a microplate reader, and the killing rate was calculated.

Killing rate=(Reading of Target only−Reading of Target plus Effector)/Reading of Target only.

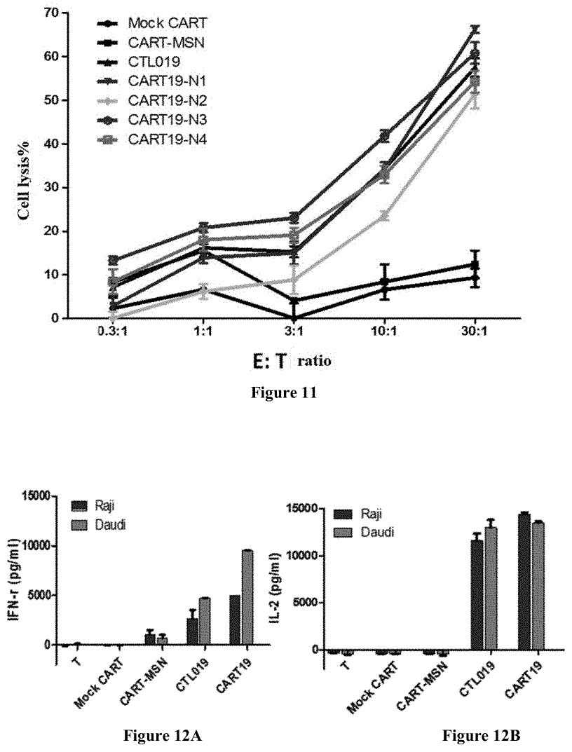

The measured killing results (FIG. 11) showed that CART19 cells had significant killing effect on leukemia tumor cells in a dose-dependent manner, and its killing ability was comparable to that of the positive control CTL-019.

### Test Example 10. Release of Cytokines During Killing of Target Cells by CART19 Cells In Vitro

CART19-N2 cells were incubated with Raji tumor target cells and Daudi target cells for 4 h (E:T=5:1), respectively, and 150 μl of supernatant was taken for the measurement of the concentration of IL-2 and IFN-γ in the supernatant by using BD Human IL-2 ELISA Kit II (Cat. No. 550611), and Human IFN-γ ELISA Kit II (Cat. No. 550612). Results were shown in FIG. 12.

The results showed that both CTL-019 and CART19-N2 cells produced a large number of cytokines IFN-γ and IL-2 when being co-cultured with target cells, indicating that both CTL-019 and CART19-N2 can exhibit characteristics of killing T cells. Moreover, CART19-N2 cells released more IFN-γ compared to CTL-019 cells, indicating that CART19-N2 cells have stronger killing effect on target cells.

### Test Example 11. Detection of Ability of CART19 and UCART19 Cells in Killing Hematologic Cancer Cell In Vitro

Target cells (Daudi, Raji and Nalm6 cells, all purchased from ATCC) were adjusted to a cell density of 5×10⁵/ml, 100 μl was plated into a 96-well round bottom plate, then added with effector cells: T cells, CART19-N2, UCART^{TCR-/-} and UCART^{TCR-/-B2M-/-} at a range of ratio of effector cell to target cell (E:T ratio=30:1 to 1:1; 30:1, 10:1, 3:1, and 1:1), and mixed by pipetting. Centrifugation was performed at 1000 rpm for 2 min, cell lysis was detected after incubation in an incubator for 4 h. 150 μl of the supernatant was collected and frozen at −20° C. for subsequent experiments. 50 μl of supernatant was maintained in each well, added with 100 μl of detection solution (Steady-Glo® Luciferase Assay System, E2520, Promega), incubated at room temperature for 5 min, and 100 μl was pipetted into a black plate. The bioluminescence value was measured by using a microplate reader, and the killing rate was calculated.

Killing rate=(Reading of Target only−Reading of Target plus Effector)/Reading of Target only.

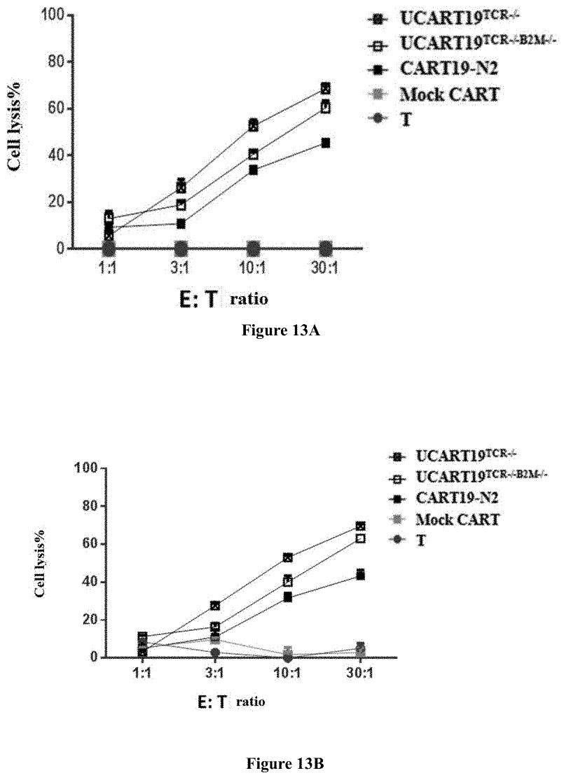

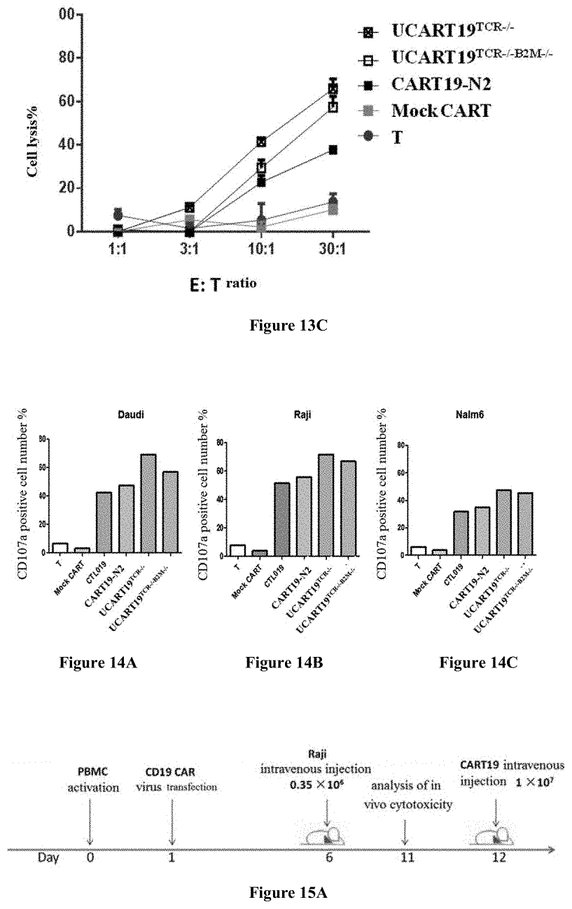

The measured killing results were shown in FIGS. 13A-13C. It can be seen from the results that CART19-N2 cells and UCART19 cells had a dose-dependent killing activity on hematological tumor cells, and the UCART^{TCR-/-B2M-/-} and UCART^{TCR-/-} cells had higher effect on killing tumor cells when compared to that of CART19.

### Test Example 12. Determination of the Expression Level of CD107a on the Cell Surface During the Killing of Target Cells by CART19 and UCART19 Cells In Vitro

Target cells (Daudi, Raji and Nalm6 cells, all purchased from ATCC) were adjusted to a cell density of 5×10⁵/ml, 100 μl of each was plated into a 96-well round bottom plate, then added with effector cells CTL-019, CART19-N2, UCART19^{TCR-/-} and UCART19^{TCR-/-B2M-/-} at a ratio of effector cell to target cell (E:T ratio=10: 1), and mixed by pipetting. Centrifugation was performed at 1000 rpm for 2 min, the cells were incubated in an incubator for 4 hours, and the cells were stained with anti-human CD8 and anti-human CD107a antibody. The ratio of CD107a-positive cells was measured by flow cytometry. Results were shown in FIGS. 14A to 14C.

From the results, it can be seen that CART19-N2 cells and UCART19^{TCR-/-} and UCART19^{TCR-/-B2M-/-} all showed significant up-regulated expression of CD107a, and such expression was significantly higher than that of CTL-019 cells, indicating that the killing CART cells, rather than helper CART cells, mainly contribute to the killing effect during the killing by CART cells.

### Test Example 13. Analysis of In Vivo Antitumor Activity CART19 Cells in Mice

Raji-luciferase cells were constructed as follows: The gene sequence of luciferase was constructed into the pLVX-EF1 viral vector, packaged into a lentivirus and then transfected into Raji cells (purchased from ATCC). The Raji-luciferase positive cells were sorted by Flow Cell Sorter. The cells were expanded and cultured for use.

NOG mice (purchased from Beijing Vital River Laboratory Animal Technologies Co. Ltd), female, 6-8 weeks, feeding environment: SPF level. One week after adaptive feeding, the mice were randomly divided into 6 groups, 6 mice per group. Each mouse was injected intravenously with 3.5×10⁵ Raji-luciferase tumor cells, and the bioluminescence intensity of tumor cells was recorded 7 days later. Each mouse was injected with 1×10⁷ CART cells. The bioluminescence intensity of Raji-luciferase cells in mice was recorded every week by using PE Small Animal Imager. The effects of different CARTs on killing Raji tumor cells in vivo were compared. The grouping of NOG mice and the injection of CART19 cells were as follows:

TABLE 4

| | Cell types | Number of cells | Number |
|---|---|---|---|
| Group | for injection | for injection | of mice |
| 1 | CART19-N1 | 1 × 10⁷ | 6 |
| 2 | CART19-N2 | 1 × 10⁷ | 6 |
| 3 | CART19-N3 | 1 × 10⁷ | 6 |
| 4 | CTL-019 | 1 × 10⁷ | 6 |
| 5 | Mock CART (vehicle) | 1 × 10⁷ | 6 |
| 6 | PBS | no | 6 |

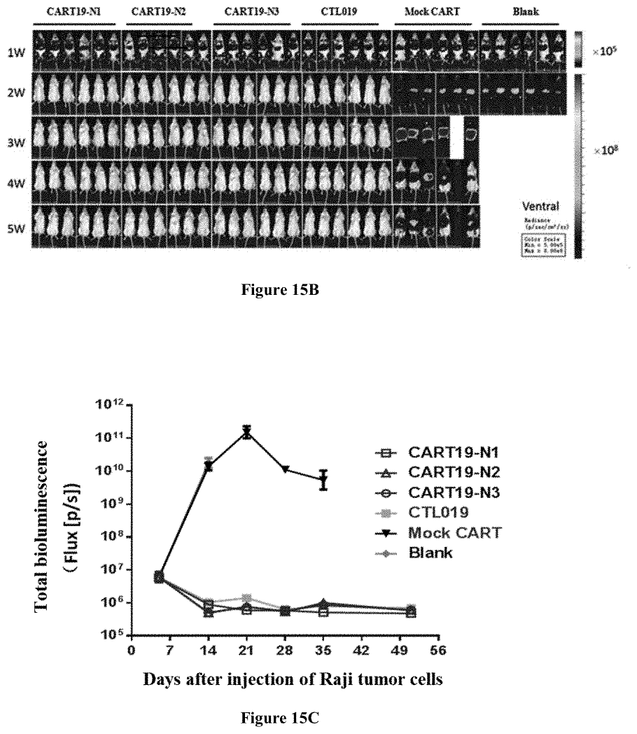

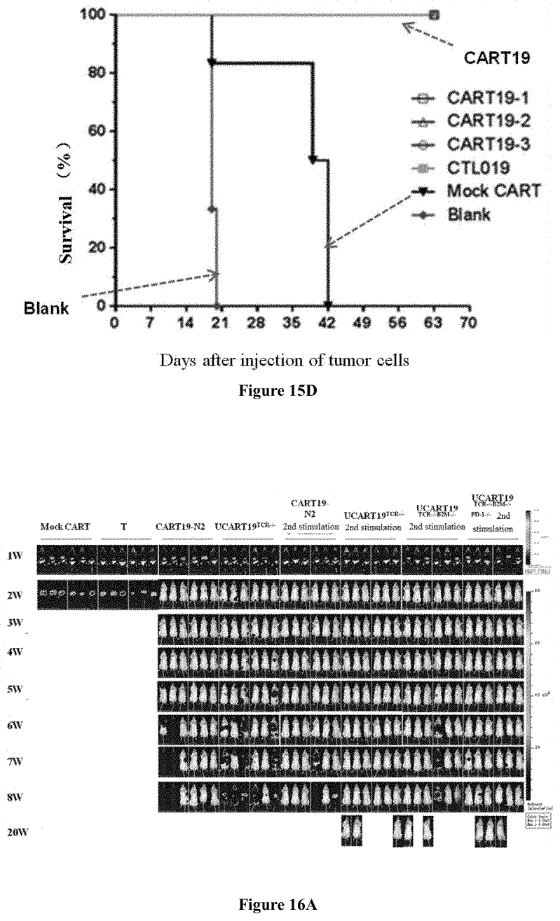

Five weeks after the establishment of mouse model and injection, the results of experimental photographs and statistical bioluminescence intensities were shown in FIGS. 15A-15B, and the overall survival rate was shown in FIG. 15C. It can be seen from the figures that CART19-N1, CART19-N2 and CART19-N3 has similar anti-tumor effects to Novartis CTL-019, and all can effectively kill leukemia tumor Raji cells in vivo.

### Test Example 14. Analysis of Antitumor Activity of CART19 and UCART19 Cells in Mice after Stimulation with K562 Cells

CART19 and UCART19 cells were cultured for 12 days and stimulated with K562-CD19 cells. 1×10⁸ K562-CD19 cells were washed once with 1640+10% FBS, and then resuspended in 10 ml of 1640+10% FBS. 25 μl of mytomycin (20 mg/ml, R&D, Cat. No. 3258) was added at 1:400 to a final concentration of 50 g/ml, and incubated at 37° C. for 30 min. After centrifuging, the supernatant was discarded and the cells were washed three times with 15 ml of 1640+10% FBS, and the supernatant was thoroughly removed at the last time. The cell density was adjusted to 1×10⁸/ml by adding 1 ml of X-VIVO medium containing 100 IU/ml of rIL-2. Mytomycin-treated K562-CD19 cells were added at a ratio of CAR-T: K562-CD19=5:1, and incubated in an incubator at 37° C. for further culture.

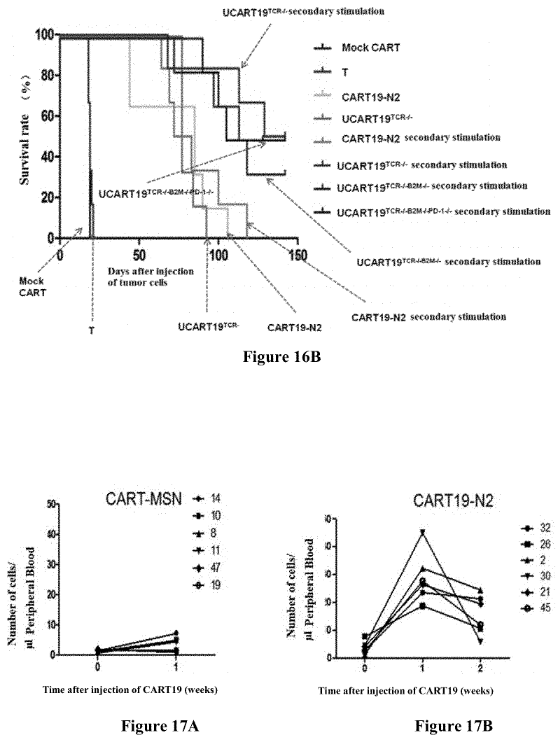

Method for establishment of model and in vivo injection was as follows: NOG mice (purchased from Beijing Vital River Laboratory Animal Technologies Co. Ltd, female, 6-8 weeks), feeding environment: SPF level. One week after adaptive feeding, the mice were randomly divided into 8 groups, 6 animals per group. Each mouse was injected intravenously with 3.5×10⁵ Raji-luciferase tumor cells for establishing NOG mouse model. After 7 days, 1×10⁷ CART19 cells were injected into each mouse, and the bioluminescence intensity of Raji-luciferase cells in mice was recorded every week by using PE Small Animal Imager after injection of CART cells. The effects of different CARTs on killing Raji tumor cells in vivo were compared. The effects of different CART19s on killing Raji tumor cells in vivo were compared. Five weeks after the establishment of mouse model and injection, the results of experimental photographs and statistical bioluminescence intensities were shown in FIG. 16A, and the overall survival rate was shown in FIG. 16B. The grouping of NOG mice was as follows:

TABLE 5

| | | Number of cells | Number |
|---|---|---|---|
| Group | Cell types for injection | for injection | of mice |
| 1 | Mock (vehicle) | 1 × 10⁷ | 6 |
| 2 | T cells | 1 × 10⁷ | 6 |
| 3 | CART19-N2 | 1 × 10⁷ | 6 |
| 4 | UCART19^{TCR-/-} | 1 × 10⁷ | 6 |
| 5 | UCART^{TCR-/- B2M-/-PD-1-/-} | 1 × 10⁷ | 6 |
| 6 | CART19-N2 undergoing | 1 × 10⁷ | 6 |
| | secondary stimulation | | |
| 7 | UCART19^{TCR-/-} undergoing | 1 × 10⁷ | 6 |
| | secondary stimulation | | |
| 8 | UCART19^{TCR-/- B2M-/-} | 1 × 10⁷ | 6 |
| | undergoing secondary stimulation | | |
| 9 | UCART^{TCR-/- B2M-/-PD-1-/-} | 1 × 10⁷ | 6 |
| | undergoing secondary stimulation | | |

From the results, it was found that the injection of CART19 cells and UCART19^{TCR-/-} cells can both significantly prolong the survival of the mouse model; the mice injected with UCART19^{TCR-/-} cells (secondarily stimulated with K562-CD19 cells) had a longer survival compared to those injected with CART19 cells, see FIG. 16B.

### Test Example 15. Analysis of In Vivo Proliferation of CART Cells in Mice

NOG mice (purchased from Beijing Vital River Laboratory Animal Technologies Co. Ltd, female, 6-8 weeks), feeding environment: SPF level. One week after adaptive feeding, the mice were randomly divided into 8 groups, 6 animals per group. Each mouse was injected intravenously with 3.5×10⁵ Raji tumor cells (purchased from ATCC) for the establishment of model. After 6 days, each mouse was injected with 1×10⁷ CART19-N2 cells and negative control CART-MSN cells. On the next day after injection of CART cells, blood was taken from mouse eye, and the number of CART cells in the peripheral blood of the mice was measured by using anti-human CD45 antibody (purchased from BD, Cat. No. 557748). The measure was performed every week (7-day interval from the previous blood collection) thereafter. Within 3 weeks after injection, the results of changes in the number of human T cells in peripheral blood of mice were shown in FIGS. 17A-17B. The numbers in the figure represent mouse numbers and groups were as follows:

TABLE 6

| Group | 1 | 2 |
|---|---|---|
| Cell types for injection | CART-MSN | CART19-N2 |
| Number of cells for injection | 1 × 10⁷ | 1 × 10⁷ |
| NOG No. | 6 | 6 |
| Mouse No. | 14, 10, 8, 11, 47, 19 | 32, 26, 2, 30, 21, 45 |

As can be seen from the results, human CART19-N2 cells were significantly expanded in mice, after CART19-N2 cells were injected into mice carrying Raji tumor cells. However, the number of CART-MSN cells did not change significantly, indicating that CART19-N2 cells were specifically stimulated by Raji tumor cells in mice and exhibited amplification signals.

### Test Example 16. Determination of Alloreactivity of T Cells Having Knockout of TCR

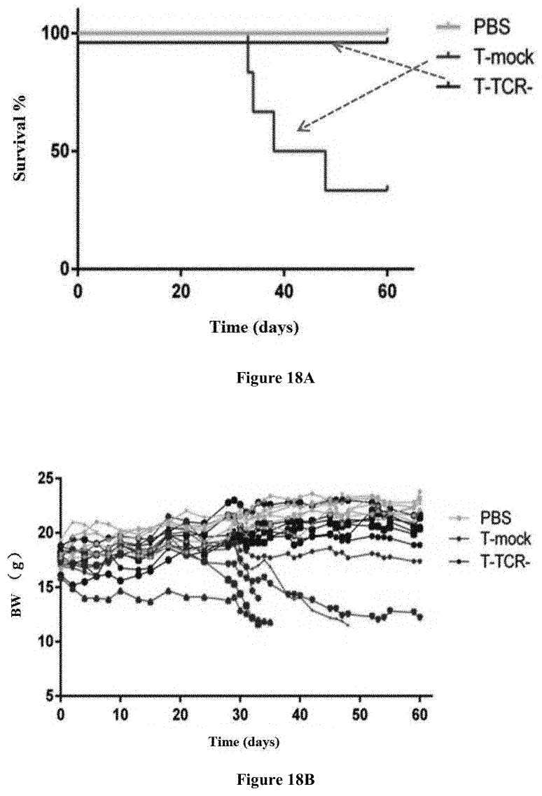

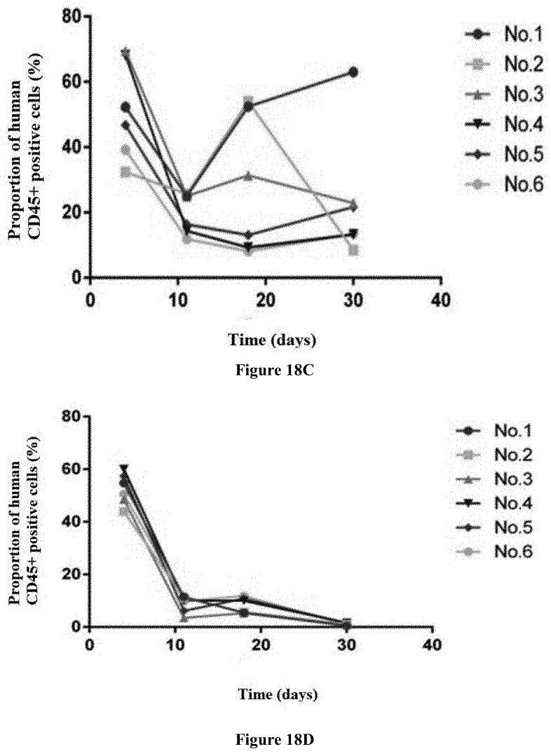

NOG mice (purchased from Beijing Vital River Laboratory Animal Technologies Co. Ltd, female, 6-8 weeks), feeding environment: SPF level. After one week of adaptive feeding, the mice were randomly divided into 5 groups, 6 animals per group. The mice were irradiated with a dose of 1 Gy from the irradiator. On day 3, the mice were injected into tail vein with PBS, 1×10⁷ TCR knockout human T cells (T-TCR⁻), 1×10⁷ mock TCR knockout human T cells (T-mock), 1×10⁷ CTL-019 cells and 1×10⁷ TCR knockout CTL-019^{TCR-/-} cells, respectively. For the preparation and screening procedures, see Example 5, Example 6 and Example 7. After 5 days, the mice were weighed every other day. Blood was taken from the fundus venous plexus of mice every week after the injection, and the number of human CD45-positive T cells in the peripheral blood of mice was measured. The groups of mice were shown in the table below. The results of the survival, body weight, and the number of human CD45-positive T cells in vivo were shown in FIGS. 18A to 18D.

TABLE 7

| | Group | | | | |
|---|---|---|---|---|---|
| | PBS | T-mock | T-TCR⁻ | CTL-019 | CTL-019^{TCR-/-} |
| | group | group | group | group | group |
| Cell types for | PBS | T-mock | T-TCR⁻ | CTL-019 | CTL-019^{TCR-/-} |
| injection | | | | | |
| Number of | none | 1 × 10⁷ | 1 × 10⁷ | 1 × 10⁷ | 1 × 10⁷ |
| cells for | | | | | |
| injection | | | | | |
| NOG No. | 6 | 6 | 6 | 6 | 6 |
| Mouse No. | 1, 2, 3, | 1, 2, 3, | 1, 2, 3, | 1, 2, 3, | 1, 2, 3, |
| | 4, 5, 6 | 4, 5, 6 | 4, 5, 6 | 4, 5, 6 | 4, 5, 6 |

The results showed that the mice injected with T-TCR⁻ cells had similar survival rate compared to that of the mice injected with PBS, both were significantly higher than that of the mice injected with human T-mock cells; and the body weight of the mice injected with human T-mock cells was decreased significantly, while the mice injected with T-TCR⁻ and PBS did not show significant body weight loss. The T cells in mice in the T-mock group were significant increased than those in the T-TCR⁻ group, indicating that TCR knockout can reduce the GvHD effect in mice. The proportion of human CD45⁺ cells in blood of the mice injected with CTL-019^{TCR-/-} was significantly decreased compared to that of the CTL-019 group, in which TCR was not knocked out, further indicating that TCR knockout can reduce the GvHD effect in mice.

1. An isolated chimeric antigen receptor, comprising a CD19 antigen binding domain, wherein the CD19 antigen binding domain comprises the amino acid sequence shown in SEQ ID NO: 18, SEQ ID NO: 20, SEQ ID NO: 22 or SEQ ID NO: 24.

2. The isolated chimeric antigen receptor according to claim 1, further comprising a co-stimulatory signaling region and/or a CD3ζ signaling domain.

3. The isolated chimeric antigen receptor according to claim 2, wherein the co-stimulatory signaling region comprises an intracellular domain of a co-stimulatory molecule selected from the group consisting of CD27, CD28, 4-1BB, OX40, CD30, CD40, PD-1, ICOS, lymphocyte function associated antigen-1, CD2, CD7, LIGHT, NKG2C, B7-H3 and any combination thereof, preferably 4-1BB co-stimulatory signaling region shown in amino acid sequence SEQ ID NO: 12.

4. The isolated chimeric antigen receptor according to claim 2, wherein the CD3ζ signaling domain comprises the amino acid sequence shown in SEQ ID NO: 14 or SEQ ID NO: 57.

5. The isolated chimeric antigen receptor according to claim 2, further comprising an extracellular hinge domain, preferably, the extracellular hinge domain comprises the human CD8α leading signal region as shown in amino acid sequence SEQ ID NO: 6 and the human CD8α hinge region as shown in amino acid sequence SEQ ID NO: 8.

6. The isolated chimeric antigen receptor according to claim 5, further comprising a CD8α transmembrane domain, preferably, the amino acid sequence of CD8α transmembrane domain is shown in SEQ ID NO: 10.

7. The isolated chimeric antigen receptor according to claim 1, comprising the amino acid sequence shown in SEQ ID NO: 26, SEQ ID NO: 28, SEQ ID NO: 30 or SEQ ID NO: 32.

8. An isolated nucleic acid molecule encoding the chimeric antigen receptor according to claim 1.

9. The isolated nucleic acid molecule of claim 8, comprising the nucleic acid sequence shown in SEQ ID NO: 17, SEQ ID NO: 19, SEQ ID NO: 21, or SEQ ID NO: 23.

10. The isolated nucleic acid molecule according to claim 9, comprising a nucleic acid sequence encoding co-stimulatory signaling region and/or a nucleic acid sequence encoding CD3ζ signaling domain, preferably, the nucleic acid sequence encoding co-stimulatory signaling region is shown in SEQ ID NO: 11, and the nucleic acid sequence encoding CD3ζ signaling domain is shown in SEQ ID NO: 13 or SEQ ID NO: 56.

11. The isolated nucleic acid molecule of claim 10, further comprising a nucleic acid sequence encoding extracellular hinge domain, preferably, the nucleic acid sequence encoding extracellular hinge domain comprises the human CD8α leading signal region shown in SEQ ID NO: 5 and the human CD8α hinge region shown in SEQ ID NO: 7.

12. The isolated nucleic acid molecule of claim 11, further comprising a CD8α transmembrane domain shown in SEQ ID NO: 9.

13. The isolated nucleic acid molecule according to claim 8, comprising a nucleic acid sequence encoding CD19 chimeric antigen receptor shown in SEQ ID NO: 25, SEQ ID NO: 27, SEQ ID NO: 29 or SEQ ID NO: 31.

14. A vector comprising the isolated nucleic acid molecule of claim 8; preferably, the vector is selected from the group consisting of DNA, RNA, plasmid, lentiviral vector, adenoviral vector, and retroviral vector; most preferably, the vector further comprises a promoter, preferably EF-1 promoter shown in SEQ ID NO: 4.

15. A T cell, which comprises the isolated nucleic acid molecule according to claim 8.

16. The T cell according to claim 15, which is a modified T cell and comprises a nucleic acid capable of down-regulating endogenous gene(s) of the T cell, wherein the endogenous gene(s) is (are) any one or more selected from the group consisting of TRAC, TRBC, B2M and PD-1 genes.

17. The T cell according to claim 16, wherein the nucleic acid capable of down-regulating endogenous gene(s) of the T cell is selected from the group consisting of antisense RNA, antigomer RNA, siRNA, shRNA and CRISPR-Cas9 system, preferably CRISPR-Cas9 system;
wherein the CRISPR-Cas9 system further comprises sgRNA(s) targeting the endogenous gene coding sequence(s) or expression-regulating sequence(s) thereof of the T cell, wherein the sgRNA(s) is/are formed by linking, from 5′ to 3′, a crRNA targeting the endogenous gene(s) with 17 nt, 18 nt, 19 nt or 20 nt in length to a tracrRNA corresponding to the Cas9 protein.

18. The T cell according to claim 17, wherein the endogenous gene(s) is(are) one or more selected from the group consisting of TRAC, B2M and PD-1, wherein the crRNA(s) that target the endogenous gene TRAC is (are) any one or more crRNAs selected from the group consisting of SEQ ID NOs: 37, 38, 39, 40, 41, 42, 43, 44, 45, 46, 47 and 48, preferably SEQ ID NO: 47; the crRNA that targets the endogenous gene B2M is shown in SEQ ID NO: 49; and the crRNA(s) that target the endogenous gene PD-1 is (are) any one or more crRNAs selected from the group consisting of SEQ ID NOs: 50, 51 and 52, preferably SEQ ID NO: 52.

19. The T cell according to claim 18, wherein the crRNA that down-regulates the endogenous gene TRAC is shown in SEQ ID NO: 47, the crRNA that down-regulates the endogenous gene B2M is shown in SEQ ID NO: 49, and the crRNA that down-regulates the endogenous gene PD-1 is shown in SEQ ID NO: 52; and
the chimeric antigen receptor comprises the amino acid sequence shown in SEQ ID NO: 26, SEQ ID NO: 28, SEQ ID NO: 30 or SEQ ID NO: 32, and preferably comprises the amino acid sequence shown in SEQ ID NO: 28.

20. A method for preparing the T cell according to claim 16, including:
a) introducing a nucleic acid encoding a chimeric antigen receptor into the T cell; and
b) introducing a sgRNA nucleic acid into the T cell via the CRISPR-Cas9 system, said sgRNA nucleic acid is capable of down-regulating the expression of endogenous target gene(s) of the T cell.

21. A pharmaceutical composition comprising
the isolated chimeric antigen receptor according to claim 1,
and, a pharmaceutically acceptable carrier, diluent or excipient.

22. A method of treating or preventing tumor disease, autoimmune disease, or infectious disease caused by virus or bacteria in a subject in need thereof, the method comprising administering an effective amount of the pharmaceutical composition according to claim 21 to the subject.

23. The method according to claim 22, wherein the tumor disease is CD19-mediated cancer, preferably selected from the group consisting of breast cancer, prostate cancer, ovarian cancer, cervical cancer, skin cancer, pancreatic cancer, colorectal cancer, kidney cancer, liver cancer, brain cancer, lung cancer, thyroid cancer and hematological cancer; more preferably is hematological cancer, wherein the hematological cancer is selected from the group consisting of acute leukemia including acute lymphocytic leukemia, acute myelocytic leukemia, acute myelogenous leukemia and myeloblastic leukemia, promyelocytic leukemia, myelomonocytic leukemia, monocytic leukemia and erythroleukemia; and chronic leukemia including chronic myelocytic (granulocytic) leukemia, chronic myelogenous leukemia and chronic lymphocytic leukemia and refractory CD19⁺ leukemia and lymphoma; polycythemia vera, lymphoma, mantle cell lymphoma, diffuse large B-cell lymphoma, Hodgkin's disease, non-Hodgkin's lymphoma, multiple myeloma, Waldenstrom's macroglobulinaemia, heavy chain disease, myelodysplastic syndrome, hairy cell leukemia and myelodysplasia; most preferably acute lymphocytic leukemia or chronic lymphocytic leukemia.


SEQUENCE LISTING (57 sequences)

SEQ ID NO: 1 - DNA, 22 bp, Artificial Sequence
  misc_feature: sequencing primer PLVX-PF
cattcgattagtgaacggatct

SEQ ID NO: 2 - DNA, 18 bp, Artificial Sequence
  misc_feature: sequencing primer PCDHI-R
gacggcaatatggtggaa

SEQ ID NO: 3 - DNA, 21 bp, Artificial Sequence
  misc_feature: sequencing primer Xd-SR
aaagccatacgggaagcaata

SEQ ID NO: 4 - DNA, 544 bp, Artificial Sequence
  promoter: EF1 promoter
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 5 - DNA, 63 bp, Artificial Sequence
  gene: Nucleotide sequence of human CD8-alpha leading signal region
atggccttaccagtgaccgccttgctcctgccgctggccttgctgctccacgccgccaggccg

SEQ ID NO: 6 - protein, 21 aa, Artificial Sequence
  DOMAIN: Amino acid sequence of human CD8-alpha leading signal region
MALPVTALLLPLALLLHAARP

SEQ ID NO: 7 - DNA, 135 bp, Artificial Sequence
  gene: Nucleotide sequence of human CD8-alpha hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 8 - protein, 45 aa, Artificial Sequence
  DOMAIN: Amino acid sequence of human CD8-alpha hinge region
TTTPAPRPPTPAPTIASQPLSLRPEACRPAAGGAVHTRGLDFACD

SEQ ID NO: 9 - DNA, 72 bp, Artificial Sequence
  gene: Nucleotide sequence of human CD8-alpha transmembrane region
atctacatctgggcgcccttggccgggacttgtggggtccttctcctgtcactggttatcaccctttactgc

SEQ ID NO: 10 - protein, 24 aa, Artificial Sequence
  DOMAIN: Amino acid sequence of human CD8-alpha transmembrane region
IYIWAPLAGTCGVLLLSLVITLYC

SEQ ID NO: 11 - DNA, 126 bp, Artificial Sequence
  gene: Nucleotide sequence of human 4-1BB intracellular region
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 12 - protein, 42 aa, Artificial Sequence
  DOMAIN: Amino acid sequence of human 4-1BB intracellular region
KRGRKKLLYIFKQPFMRPVQTTQEEDGCSCRFPEEEEGGCEL

SEQ ID NO: 13 - DNA, 336 bp, Artificial Sequence
  gene: Nucleotide sequence of human CD3-zeta intracellular region
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 112 aa, Artificial Sequence
  DOMAIN: Amino acid sequence of human CD3-zeta intracellular region
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 120 aa, Artificial Sequence
  DOMAIN: Heavy chain variable region amino acid sequence of FMC63 antibody
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 16 - protein, 120 aa, Artificial Sequence
  DOMAIN: Light chain variable region amino acid sequence of FMC63 antibody
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPP

SEQ ID NO: 17 - DNA, 726 bp, Artificial Sequence
  gene: Nucleotide sequence of CD19-N1scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 18 - protein, 242 aa, Artificial Sequence
  DOMAIN: Amino acid sequence of CD19-N1scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 19 - DNA, 732 bp, Artificial Sequence
  gene: Nucleotide sequence of CD19-N2 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 20 - protein, 244 aa, Artificial Sequence
  DOMAIN: Amino acid sequence of CD19-N2 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 21 - DNA, 738 bp, Artificial Sequence
  gene: Nucleotide sequence of CD19-N3 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggcggccgcaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 22 - protein, 246 aa, Artificial Sequence
  DOMAIN: Amino acid sequence of CD19-N3 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRAAAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - DNA, 765 bp, Artificial Sequence
  gene: Nucleotide sequence of CD19-N4 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 24 - protein, 255 aa, Artificial Sequence
  DOMAIN: Amino acid sequence of CD19-N4 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - DNA, 1464 bp, Artificial Sequence
  gene: Nucleotide sequence of CD19 CAR-N1
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 26 - protein, 486 aa, Artificial Sequence
  DOMAIN: Amino acid sequence of CD19 CAR-N1
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1470 bp, Artificial Sequence
  gene: Nucleotide sequence of CD19 CAR-N2
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 28 - protein, 488 aa, Artificial Sequence
  DOMAIN: Amino acid sequence of CD19 CAR-N2
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1476 bp, Artificial Sequence
  gene: Nucleotide sequence of CD19 CAR-N3
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggcggccgcaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 30 - protein, 490 aa, Artificial Sequence
  DOMAIN: Amino acid sequence of CD19 CAR-N3
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRAAAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 1503 bp, Artificial Sequence
  gene: Nucleotide sequence of CD19 CAR-N4
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 32 - protein, 499 aa, Artificial Sequence
  DOMAIN: Amino acid sequence of CD19 CAR-N4
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1461 bp, Artificial Sequence
  gene: CD19-CAR nucleotide sequence of the positive control CTL109
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 34 - protein, 486 aa, Artificial Sequence
  DOMAIN: CD19-CAR amino acid sequence of the positive control CTL109
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 2019 bp, Artificial Sequence
  gene: Nucleotide sequence of MSN-CAR (SS1CAR)
atggccttaccagtgaccgccttgctcctgccgctggccttgctgctccacgccgccaggccgcaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggtggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaaagcagcgagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatgttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaacccccaggagggcctgtataacgaactgcagaaagacaagatggccgaggcctacagcgagatcggcatgaagggcgagcggaggcggggcaagggccacgacggcctgtaccagggcctgagcaccgccaccaaggatacctacgacgccctgcacatgcaggccctgccccccaga

SEQ ID NO: 36 - protein, 673 aa, Artificial Sequence
  DOMAIN: Amino acid sequence of MSN-CAR (SS1CAR)
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - RNA, 18 nt, Artificial Sequence
  unsure: TRAC crRNA-1
caaaugugucacaaagua

SEQ ID NO: 38 - RNA, 18 nt, Artificial Sequence
  unsure: TRAC crRNA-2
aaaacugugcuagacaug

SEQ ID NO: 39 - RNA, 18 nt, Artificial Sequence
  unsure: TRAC crRNA-3
ucaagagcaacagugcug

SEQ ID NO: 40 - RNA, 18 nt, Artificial Sequence
  unsure: TRAC crRNA-4
caccuucuuccccagccc

SEQ ID NO: 41 - RNA, 18 nt, Artificial Sequence
  unsure: TARC crRNA-5
gaauaaugcuguuguuga

SEQ ID NO: 42 - RNA, 18 nt, Artificial Sequence
  unsure: TRAC crRNA-6
gauuuagagucucucagc

SEQ ID NO: 43 - RNA, 18 nt, Artificial Sequence
  unsure: TRAC crRNA-7
acggcagggucaggguuc

SEQ ID NO: 44 - RNA, 18 nt, Artificial Sequence
  unsure: TRAC crRNA-8
guuccugugaugucaagc

SEQ ID NO: 45 - RNA, 18 nt, Artificial Sequence
  unsure: TARC crRNA-9
ucaaaaccugucagugau

SEQ ID NO: 46 - RNA, 18 nt, Artificial Sequence
  unsure: TRAC crRNA-10
gaauccuccuccugaaag

SEQ ID NO: 47 - RNA, 17 nt, Artificial Sequence
  unsure: TRAC crRNA-11
gguacacggcaggguca

SEQ ID NO: 48 - RNA, 20 nt, Artificial Sequence
  unsure: TRAC crRNA-12
gagaaucaaaaucggugaau

SEQ ID NO: 49 - RNA, 18 nt, Artificial Sequence
  unsure: B2M crRNA-13
guagcgcgagcacagcua

SEQ ID NO: 50 - RNA, 20 nt, Artificial Sequence
  unsure: PD-1 crRNA-14
cgacuggccagggcgccugu

SEQ ID NO: 51 - RNA, 18 nt, Artificial Sequence
  unsure: PD-1 crRNA-15
gugcuacaacugggcugg

SEQ ID NO: 52 - RNA, 20 nt, Artificial Sequence
  unsure: PD-1 crRNA-16
ggcgcccuggccagucgucu

SEQ ID NO: 53 - RNA, 80 nt, Artificial Sequence
  unsure: tracrRNA sequence corresponding to Cas9 protein
guuuuagagcuagaaauagcaaguuaaaauaaggcuaguccguuaucaacuugaaaaaguggcaccgagucggugcuuuu

SEQ ID NO: 54 - protein, 1378 aa, Artificial Sequence
  DOMAIN: Amino acid sequence of Cas9 protein (including NLS)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVMDK

SEQ ID NO: 55 - DNA, 127 bp, Artificial Sequence
  unsure: Exemplary N-oligo sequence
tcatgtggtcggggtagcggctgaagcactgcacgccgtacgtcagggtggtcacgagggtgggccagggcacgggcagcttgccggtggtgcagatgaacttcagggtcagcttgccgtaggtggc

SEQ ID NO: 56 - DNA, 336 bp, Artificial Sequence
  gene: Nucleotide sequence of CD3-zeta intracellular region
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 57 - protein, 112 aa, Artificial Sequence
  DOMAIN: Amino acid sequence of CD3-zeta intracellular region
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR